(19) United States
(12) Patent Application Publication
(10) Pub. No.: US 2005/0192757 A1
(43) Pub. Date: Sep. 1, 2005

(54) Method of constructing teritiary structure of protein composed of plural chains

(75) Inventors: Hideaki Umeyama, Urayasu-shi (JP); Mitsuo Iwadate, Hanyu-shi (JP); Eiichiro Suzuki, Sagamihara-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Hideaki Umeyama, Urayasu-shi (JP)

(21) Appl. No.: 10/887,104

(22) Filed: Jul. 9, 2004

Related U.S. Application Data
(63) Continuation of application No. PCT/JP03/00057, filed on Jan. 8, 2003.

(30) Foreign Application Priority Data
Jan. 9, 2002 (JP) 2002-002859

(51) Int. Cl. G06F 19/00; G01N 33/48; G01N 33/50

(52) U.S. Cl. 702/19

(57) ABSTRACT

A method is provided of constructing a tertiary structure of a protein composed of plural chains having given arbitrary amino acid sequences by extending an comparative modeling method of constructing a tertiary structure of a protein composed of a single chain having a given arbitrary amino acid sequence (extended modeling method). In this method, an input file format of the plural chains in a computer software program is each corrected so as to present a form of a temporary single chain (correction of sequence alignment) and the tertiary structure is constructed based on the modeling method while assuming that the structure has plural chains in calculation of a potential formula by the computer software program, thereby constructing the tertiary structure of the target protein. Namely, a method is provided of constructing the tertiary structure of an arbitrary protein having plural chains, which serves as a particularly important key factor in developing drugs or the like, highly accurately and much more efficiently than by a conventional method.

### CROSS-REFERENCE TO RELATED APPLICATIONS

The present application is a continuation of PCT/JP03/00057 filed on Jan. 8, 2003, which claims priority to JP 2002-002859, filed on Jan. 9, 2002, which are incorporated herein by reference in their entirety.

### TECHNICAL FIELD

The present invention relates to a method of constructing a tertiary structure of a protein composed of plural chains, more specifically, to a method of predicting a tertiary structure of a protein composed of plural chains whose tertiary structure is not known. According to this method, a protein composed of plural chains is treated as a temporary single chain for simplification, thereby the protein structure can be predicted with consideration given to the interaction between the plural peptide chains constituting the protein. As a result, as will be described in the examples mentioned below, more highly reliable prediction of a tertiary structure of a protein can be performed than a conventional method.

In addition, the present invention relates to a tertiary structure model of a protein obtained by the method, an available database for the method, a database structure, a computer software program, a computer installed therewith, an interface and the like.

For example, when a stop signal of a delimiter is added to each C terminal residue of an amino acid sequence composed of plural chains, and a protein tertiary structure composed of three-dimensional coordinates of a main-chain and a side-chain is constructed, potential calculation can be performed with consideration given to the interaction of the amino acid residues between the protein chains by obtaining the C terminal residue number of each chain based on this stop signal. According to this method, a model excellent in packing of the side-chain can be constructed, therefore, the tertiary structure of a protein composed of plural chains can be predicted with higher reliability than by a conventional method. Addition of the delimiter is performed for handling the plural chains as a single chain by binding all the plural chains one another. Therefore, it is not necessary to add the delimiter to the C terminal end of the last binding chain when making it a single chain (the last terminal end when making it a single chain).

In the present invention, a chain which constitutes or can constitute a protein such as a polypeptide chain or a ligand, namely, a chain of the protein is referred to as merely “a protein chain” in some cases.

In the case where a chain (protein chain) other than a polypeptide chain exists in a protein, for example, a component which is one protein chain constituting plural chains is a low molecular weight ligand such as a peptide, various binding states can be created by mutating arbitrarily the amino acid sequences of the ligand.

As a potential parameter, by adding an arbitrary interatomic potential parameter to an interatomic parameter constituting amino acid residues, modification into an arbitrary ligand molecule can be performed. In addition, by fixing the amino acid sequences of the ligand and creating a data set in which the amino acid sequences of an environmental protein chain has been mutated diversely, various receptor models, which can bind to a specific ligand, can be constructed. Also, since the interaction between proteins composed of plural chains can be represented accurately, a model, in which a recognition region related to a function is described, can be constructed. By mutating the amino acid residues of the interaction region, a model, in which an increase or decrease in the function can be adjusted, can be constructed. In the case of a single chain, after it is divided into a domain or a module to assume it as plural chains, they are restored as a temporary single chain, thereby a highly accurate tertiary structure model can be attempted.

In the method of constructing a tertiary structure of a protein of the present invention, the basic backbone is to make the use of an comparative modeling method of proteins, in particular, a homology modeling method or a threading method. As a protein tertiary structure, the one whose three-dimensional coordinates have been determined by an X-ray crystallographic analysis or the like is used as a template to be referred to. In practical, unless a protein whose structure has been determined as plural chains is used as a template, accurate relative configuration of respective protein chains, particularly, respective polypeptide chains cannot be determined in many cases. In other words, the present invention is a comparative modeling method using a reference protein complex whose relative configuration is known. However, homology of amino acid sequences between a target protein, which is to be a subject of modeling, and a reference protein is not necessarily high, all the proteins which meet the predetermined requirement (E-value) described later can be used as a reference protein (threading method). In addition, if modeling is performed only with respect to, for example, the interaction interface, existence of an experimental structure which can be referred to a whole complex is not necessarily required.

### BACKGROUND ART

Determination of genome sequences has been performed worldwide, and lots of amino acid sequences encoded by more than 70 types of genomes have been determined in the same way. In particular, complete genome sequence analysis of human and many other living species has been proceeding to construct a database of the sequence information (refer to Gerardo Jimenez-Sanchez, Nature 409, 853-855 (2001)). Although the function of a gene can be specified or predicted from the genome sequence to a certain degree, there are so many genes whose functions cannot be predicted from only the sequence information. For a gene, the protein obtained by translating the DNA sequence of the gene into an amino acid sequence actually plays a function. For elucidation of the function of a protein, determination of the tertiary structure is performed by an comparative method such as an X-ray crystallographic analysis or NMR, however, it generally needs works requiring considerable time and effort. Therefore, a protein with a known tertiary structure whose amino acid sequences show a high homology with those of a target protein is searched, and prediction of the function of the target protein is performed.

As a method of predicting the tertiary structure of a protein itself from the amino acid sequences of a protein with an unknown tertiary structure, a homology modeling method is generally used (refer to T. Yoneda, H. Komooka and H. Umeyama, J. Protein Chem., 16, 597-605, 1997. The whole content is included for reference in this description as a part). This is a computational scientific method primarily including the following 4 steps.

(1) When an amino acid sequence of an arbitrary target protein with an unknown tertiary structure (target sequence) is given, a reference protein having a similar sequence to the target sequence (reference sequence) is selected from a tertiary structure database such as PDB (Protein Data Bank) by searching a protein whose sequence is statistically significantly similar (homology search) to give a sequence alignment between the target sequence and the reference sequence.

For performing the database search and the alignment, computer software such as FASTA (refer to Pearson W R, Methods Enzymol, 266, 227-258, 1996), PSI-BLAST (refer to Schaffer A A, Wolf Y I, Ponting C P, Koonin E V, Aravind L and Altschul S F, Bioinformatics, 12, 1000-1011, 1999) or LIBRA (refer to Ota, M. and Nishikawa, K., Protein Engineering, 10, 339-351, 1997) can be used. FASTA is a program for performing the matching of the sequences of 20 alphabetical characters which mean 20 natural amino acids. It has been reported that if a tertiary structure is constructed to a reference protein with a high homology (similarity of amino acid residues about 30% or more, which corresponds to a FASTA's E-value of about 0.01 or less), a highly reliable model can be constructed.

On the other hand, in PSI-BLAST, the matching of character sequences is performed in the same way. However, PSI-BLAST does not have information whether characters agree or not, but has a characteristic of optimizing an alignment by calculating the similarity of characters called a profile as a substitution matrix for each character sequence region of a homologous protein and further repeating the calculation. LIBRA is a program based on the 3D-1D method (also known as the threading method), and used for examining a known tertiary structure and searching a homologous sequence to a target sequence. Therefore, its search algorithm is apparently different from that of FASTA or PSI-BLAST. Accordingly, LIBRA can sometimes detect homology between sequences widely (though including also an error), unlike the case of FASTA or PSI-BLAST.

(2) When the alignment calculated by FASTA, PSI-BLAST, LIBRA or the like is used, the correspondence relationship between the target sequence and the homologous reference sequence is determined for each character sequence region. Based on this relationship, three-dimensional coordinates of amino acid residues of the target sequence are created from the three-dimensional coordinates of the reference protein.

(3) In the case where the corresponding amino acid residue in the target sequence side to the reference sequence does not exist (amino acid residue deletion), the coordinates of the amino acid residue at the position of the reference protein side are not used. On the contrary, in the case where the corresponding amino acid residue in the reference sequence side to the target sequence does not exist (amino acid residue insertion), the coordinates of the amino acid residue at the position of the target sequence is created by searching an appropriate one from the database of protein fragment coordinates which has been prepared beforehand (refer to, for example, K. Ogata and H. Umeyama, Journal of Molecular Graphics and Modeling 18, 258-272, 2000. The whole content is included for reference in this description as a part).

(4) In the construction of the protein coordinates according to the above-mentioned (2) and (3), an inappropriate gap, collision or strain may structurally occur between amino acid residues. Therefore, these unnatural structures are normalized by an energy minimization calculation or a molecular dynamics calculation (refer to M. Takeda-Shitaka, H. Umeyama, FEBS Letters, 425, 448-452, 1998. The whole content is included for reference in this description as a part).

In some modeling software, for smoothly eliminating the structural strains mentioned in (4), the calculation and search procedures mentioned in (2) to (4) are performed for all atoms of the protein by, for example, the simulated annealing (SA) method gradually, instead of simultaneously.

“SA method” is a method for finding the global minimum point of energy E without getting trapped in a local minimum point, when a new state x′ is obtained by applying a perturbation to a system in a state x, by performing transition to a new state x′ with a high probability if the energy value E(x′) in the new state is lower than the energy value E(x) in the old state, and with a low probability if it is higher. More specifically, energy minimization by the SA method is performed first for an a carbon atom which constitutes a protein backbone, subsequently for main-chain atoms including the a carbon atom, finally for all the atoms of the protein including side-chain atoms. “molecular dynamics calculation” is a method for calculating a coordinate change in which potential energy E should decrease by representing the potential energy E of a system as a coordinate function and performing an energy minimization calculation by mainly steepest descent method, the conjugate gradient method or the like. “Monte Carlo method” is a calculation method of probability dependent energy optimization based on statistical mechanics.

As mentioned above, if an alignment to the target sequence mentioned in (2) is obtained, its tertiary structure can be predicted and constructed via the creation of three-dimensional coordinates (refer to above-mentioned K. Ogata and H. Umeyama, Journal of Molecular Graphics and Modeling 18, 258-272, 2000).

However, conventionally, when an arbitrary sequence to a protein composed of plural chains is given, one or more proteins with a high homology are selected for each chain independently from a tertiary structure database such as PDB, an alignment is given, and modeling is performed independently for each of them. Therefore, as will be described in the examples mentioned below, a tertiary structure in which a protein-protein interaction mode is fully reflected could not be obtained. In particular, in the case where a ligand binding region of a drug, an enzyme substrate or the like is composed of plural chains, the above drawback is critical. In the case where for a drug or an enzyme substrate, a property such as function efficiency of a protein that accepts such a ligand is changed, the same drawback occurs. Conventionally, as a method of eliminating this drawback, there is a method of improving the drawback by presuming an interaction mode among plural chains after constructing a model by a molecular dynamics method or the like. However, an enormous amount of calculation time and computing machine resources are needed for searching a global minimum. In addition, since molecular dynamics parameters, which should originally correspond to a multibody, correspond to a two-body, it is difficult to give the native tertiary structure. Therefore, there is a problem that the method is not suitable for a genome-wide industrial application.

### DISCLOSURE OF THE INVENTION

1. Problems to be Solved by the Present Invention

The present inventors investigated a method of constructing the tertiary structure of a protein composed of plural chains by converting the plural chains to a single chain when an arbitrary sequence for the protein is given. On an experimental basis, a method of binding with the appropriate number of glycine (G)—oligomer chains for converting natural or conceptional plural chains to a single chain is known as a spontaneously conceived one (refer to JP-A-2002-112782). In the calculation, the method can be handled in the same way, however, this method cannot always be adopted. For example, in the case where the C terminal of the initial chain and/or the N terminal of the subsequent chain is located at the complex interface, glycine-oligomer cannot be inserted. A complex can be formed in the same method as the distance geometry method based on the distance information of NMR. However, this method requires a lot of input data, thereby the preparation of calculation is complicated. Accordingly, a simple method of constructing a structure has been awaited.

It is an object that the present invention, with taking the above-mentioned situation into consideration, to provide a method of constructing a tertiary structure of an arbitrary protein composed of plural chains, which serves as a particularly important key factor in developing drugs or the like, highly accurately and much more efficiently than by a conventional method. Another object of the present invention is to provide a method of performing various modifications of a ligand molecule or a modification of a protein such as a receptor promptly and efficiently. Another object of the present invention is to specify the cause of an genetic disease and to promote development of drugs related to the disease by constructing a protein model composed of plural chains to elucidate a protein-protein interaction mode and to clarify the recognition mechanism of the interaction.

2. Means for Solving the Problems

The present inventors repeatedly investigated to obtain an appropriate tertiary structure model when arbitrary amino acid sequences of a protein composed of plural chains are given, and as a result, found or developed the following (1) to (10) methods and computer programs therefor.

(1) A method of fully automatically or manually constructing a tertiary structure model composed of three dimensional coordinates of main-chains and side-chains of a protein in the same way as a single chain in the case where an arbitrary amino acid sequence is given, by correcting an input file format of the plural chains in a computer program to present a form of a temporary single chain (a single chain) so as to extend an applicable range of a conventional comparative modeling method, for example, a homology modeling method from a protein composed of a single chain to a protein composed of plural chains (an extended modeling method), while taking that the protein is composed of plural chains into consideration in a potential formula, and a computer program therefor.

It is desirable that the number of chains constituting the target protein and the number of chains constituting the reference protein be the same. For example, in the case where the number of chains constituting the target protein is two, a reference protein having two chains can be adopted as a reference protein (candidate) selected for constructing the tertiary structure. Also, in the case where the number of chains constituting the target protein is three, a reference protein having three chains can be adopted as a reference protein (candidate) selected for constructing the tertiary structure. However, the numbers of chains of both (the reference protein and the target protein) are not necessarily the same, and the reference protein that includes a protein constructing the tertiary structure of the target protein or a portion (plural chains) thereof can be adopted as a reference protein.

A file format of sequences for a computer is corrected so as to regard the respective proteins composed of plural chains as a single chain (temporary single chain), however, there is no particular restriction on a method of making it a temporary single chain. For example, a method of correcting an amino acid sequence alignment between the target protein composed of plural chains and the reference protein to an alignment as a temporary single chain by inserting an identification mark at the boundary of each protein chain (e.g., polypeptide chain) is convenient and advantageous for performing the program. There is a method of being capable of converting plural chains to a temporary single chain other than this. For example, it is also feasible by a method of creating a separate file in which the boundary of each protein chain is registered sequentially with the residue number and sending it as a variable to a computer software program. Both are different only in the file format used for indicating the boundary of each protein chain, and the contents are exactly equivalent. In the same way, a method of using an identification number or the like as an internal variable indicating the boundary position or the boundary of each protein chain obtained from the result of performing sequence alignment procedure in a computer software program is also suitable. These methods can be, as a matter of course, used in the present invention. Therefore, these contents are included in the content of the above-mentioned development (content of the present invention).

In addition, by correcting the input file format of the plural chains so as to present a form of a temporary single chain, the input file format and an interaction between protein chains, which generally become more complicated with the increase in the number of chains, can be always described with the simplest input format and the clearest potential formula.

(2) In the above method, a method of correction to an sequence alignment having extensibility of the number of chains by selecting an amino acid sequence of the reference protein composed of plural chains based on a result of an alignment output preferably using FASTA, PSI-BLAST, LIBRA, FAMS, RPS-BLAST, IMPALA, ClustalW, HMMER, BIOCES or the like which is a variety of existing computer software on correction of the sequence alignment, adding a delimiter (e.g., “U” or the like) other than an amino acid symbol to next to the end (C terminal) of each amino acid sequence of the sequence alignment to take the boundary between respective chains consideration on modeling, and handling it as if it were a single chain. As mentioned above, addition of the delimiter is for enabling to handle plural chains as a single chain by binding all the chains one another. Therefore, addition of the delimiter is not needed for the C terminal end of the last binding chain when making it a single chain (the last terminal end when making it a single chain). Even if it is added, the delimiter of the last terminal end can be disregarded.

By preparing the sequence alignment having such a format, the upper limit of the chain number of a protein (currently up to 36 chains as a default) can be unlimitedly extended in theory (within the limits of the memory in a computer).

(3) In an extended modeling method such as an extended homology modeling, a method of constructing a model structure, by which construction and optimization of Cα atomic coordinates are performed or can be performed sequentially or simultaneously for all chains by determining the C terminal residue number of each protein chain (e.g. polypeptide chain) from the sequence alignment corrected by the above-mentioned method (2) when performing potential calculation, disconnecting a chemical bond potential (potential energy arising from chemical bond length) and a chemical bond angle potential (potential energy arising from chemical bond angle) at the boundary and adding an interatomic interaction potential (energy) at the boundary, and a computer program therefor.

(4) In an extended modeling method such as an extended homology modeling, a method of constructing a model structure, by which construction and optimization of coordinates of main-chain atoms of N, Cα, C and O (carbonyl oxygen) and coordinates of side-chains of amino acid residues are performed or can be performed sequentially or simultaneously for all chains by determining the C terminal residue number of each protein chain (e.g. polypeptide chain) from the sequence alignment corrected by the above-mentioned method (2) when performing potential calculation, disconnecting a chemical bond potential, a chemical bond angle potential and a chemical bond torsional angle potential at the boundary of the main-chain of each protein and adding an interatomic interactionpotential at the boundary, and a computer program therefor.

FIG. 1 shows the relationship between the whole amino acid sequences of the protein and the serial numbers (hereinafter referred to as “k_{N}”. N indicates the number of protein chains) of the C terminal residues of each protein chain (e.g. polypeptide chain). FIGS. 2 to 8 show specific descriptions of the above-mentioned methods (3) and (4). In each step of construction and optimization of Cα atomic coordinates, as shown in FIGS. 2 to 4, disconnection of the chemical bond potential and the chemical bond angle potential between the C terminal residue k_{N} of the N-th protein chain and the N terminal residue k_{N}+1 of the (N+1)-th protein chain, and addition of the interatomic interaction potential are performed sequentially or simultaneously for all protein chains from N=1 to (the total number of chains)−1 (M−1). In each step of construction and optimization of main-chain atomic coordinates, as shown in FIGS. 5 to 8, disconnection of the chemical bond potential, the chemical bond angle potential, the chemical bond torsional angle potential between the C terminal residue k_{N} of the N-th protein chain and the N terminal residue k_{N}+1 of the (N+1)-th protein chain, and addition of the interatomic interaction potential are performed sequentially or simultaneously for all protein chains from N=1 to (the total number of chains)−1 (M−1). Accordingly, validity of the model structure can be improved. In addition, simplification and improvement of efficiency of calculation steps can be realized.

(5) A method of constructing a protein-protein interaction model, when using an extended modeling method, in particular an extended homology modeling method or a threading method, by accurately determining atomic coordinates of an amino acid residue of a protein-protein interaction region.

(6) A method of constructing various peptide ligand models, which can bind to a target protein, when using an extended modeling method such as an extended homology modeling in the case where a certain protein component constituting plural chains includes a peptide ligand (including amino acid derivatives, peptide derivatives and the like), by creating a data set, in which an amino acid sequence of the ligand is mutated diversely.

(7) A method of constructing various ligand receptor (protein) models, which can bind to a specific ligand, when using an extended modeling method, in particular an extended homology modeling method, in the case where a component constituting plural chains includes a peptide ligand (including amino acid derivatives, peptide derivatives and the like), by fixing an amino acid sequence of the ligand and creating a data set, in which an amino acid sequence of an environmental protein chain is mutated diversely.

(8) A method of constructing a useful protein model, when using an extended modeling method, in particular, an extended homology modeling method, in the case where a component constituting plural chains is a common protein (chain), by creating a data set, in which an amino acid sequence of a recognition region of proteins is mutated diversely, and increasing or decreasing the function efficiency of the protein.

(9) A method of attempting a highly accurate tertiary structure model, in the case of a single chain, by dividing a protein into domains or modules to have it assumed as plural chains, and enabling to restore the plural chains to a temporary single chain by applying the above-mentioned (1) to (8).

(10) A computer software program, for enabling to browse and search the below-mentioned content for a database composed of the tertiary structure model of a protein, the ligand model and the ligand receptor (protein) model, which are constructed based on the above-mentioned (1) to (9) and a computer installed with the program.

The subjects to be browsed and searched are the following:
A) a gene identification code or a protein identification code of a target protein composed of plural chains, an about one-line function description, a target amino acid sequence and (coordinates of) three-dimensional structure constructed by the above-mentioned (1) to (9);
B) a gene identification code or a protein identification code of a reference protein, an about one-line function description, a reference amino acid sequence and (coordinates of) three-dimensional structure of the reference protein; and
C) a database structure in which an alignment result between a target sequence and a reference sequence, a homology value and an E-value are described and compiled.

Namely, the present invention, as an embodiment, lies in a method of constructing a tertiary structure of a protein composed of plural chains having given arbitrary amino acid sequences (target protein) by extending an comparative modeling method of constructing a tertiary structure of a protein composed of a single chain having a given arbitrary amino acid sequence (extended modeling method), the method comprising the steps of correcting an input file format of the plural chains in a computer software program so as to present a morphology as a temporary single chain (correction of sequence alignment) and constructing the tertiary structure based on the modeling method while assuming that the structure has plural chains in the calculation of a potential formula by the computer software program. The input file format as a temporary single chain and the handling it as plural chains in the potential formula of the present invention can be applied to an comparative modeling method other than FAMS, and also to a non-comparative modeling method.

As the comparative modeling methods, a homology modeling method (refer to K. Ogata and H. Umeyama, Journal of Molecular Graphics and Modeling 18, 258-272, 2000) and/or a threading method can be given.

In the above-mentioned methods, an objective tertiary structure can be constructed fully automatically or manually.

As a content of the correction in the above-mentioned method, selecting a sequence, which has a known structure and includes the same number of plural protein chains (such as polypeptide chains and ligands) as the target sequence, and handling it as a temporary single chain can be given. For example, in the case where the target protein is composed of polypeptide chains, it includes selecting an amino acid sequence of a reference protein composed of the same number of polypeptide chains, thereby handling the plural polypeptide chains (amino acid sequences) constituting the protein as a single chain (temporary single chain) in a form in which the N terminal end (head end) of one chain has bound to the C terminal end (tail end) of another chain sequentially. At this time, the plural polypeptide chains constituting the target sequence can also be handled as a single chain in a form in which they have bound sequentially in the same manner. For example, it is preferable that a protein can be handled as a protein of a single chain on, for example, a computer by adding a delimiter, preferably a delimiter other than amino acid symbols (e.g., a character “U”), to the C terminal end (tail end) of each amino acid sequence constituting the plural chains (correction of sequence alignment). In this case, the last chain among the plural chains binding sequentially, namely, when the protein is composed of N number of protein chains, the N-th protein chain does not need to bind to a chain furthermore, it is not necessary to add the delimiter to the C terminal end of the N-th protein chain (the last chain). Even if the delimiter is added to the last terminal end of the chain, it can be disregarded on a computer or the like. As mentioned above, by taking the boundaries between respective chains into consideration on modeling, and handling it as if it were a single chain, a sequence alignment having extensibility by the number of chains can be used.

Incidentally, there is no particular restriction on a protein chain constituting a target protein or a reference protein as long as it is recognized as a chain constituting a protein practically or it can constitute a protein. The typical examples include a polypeptide chain. “A polypeptide chain” may be a chain including primarily a polypeptide residue without being limited to a chain which is constituted only by a peptide bond (amide bond) with an amino acid and/or a derivative thereof (such as a salt or an ester derivative). Other than this, as a protein chain, a substance selected from amino acid derivatives (e.g., natural or non-natural amino acids and their derivatives), peptide derivatives, pharmaceutical substances, nucleic acids, saccharides, organic compounds such as organic metal compounds, metal oxides and their ions, and inorganic compounds such as metals and their ions is given. In such a case, this often exists or is selected as a ligand.

In the present invention, a protein used as a target protein or a reference protein is composed of plural chains. These plural protein chains include one or more polypeptide chains as mentioned above to form a protein. For example, a protein composed of only plural polypeptide chains, a protein including one or more polypeptide chains and a substance such as an amino acid derivative or a peptide derivative, which exists as, for example, a ligand (as mentioned above), as a chain can be exemplified.

In the case of a protein including plural polypeptide chains in the plural chains, these plural polypeptide chains may be heterochains or homochains. In other words, plural polypeptide chains whose amino acid sequences are exactly the same (homochain) or different from one another (heterochain) may be included.

The above-mentioned method includes a method of searching the reference protein from a tertiary structure database and performing sequence alignment between the amino acid sequence of the reference protein and the target sequence.

As a software which searches the reference protein and outputs an alignment, FAMS, FASTA, PSI-BLAST, LIBRA, RPS-BLAST, IMPALA, ClustalW, HMMER, BIOCES (refer to Protein Engineering, vol. 2, No. 5, pp 347-351, 1989) or the like can be adopted as a preferred software.

In the above-mentioned method, it is preferable that the corrected sequence alignment be treatable to a multiple alignment, in which the amino acid sequence of the same type or a different type of the reference protein is written, by using a file having a format, in which an amino acid sequence of each protein chain (e.g., polypeptide chain) has a delimiter at the C terminal end of the amino acid sequence, and specifying a reference protein ID for each alignment section delimited by the delimiter, thereby enabling to show an average structure by superposing the sequences.

In the above-mentioned method, it is preferable that Cα atomic coordinates and main-chain atomic coordinates be constructed based on the tertiary structure database, preferably PDB or the like, and/or a database, which is obtained by modifying or sorting the tertiary structure database so as to avoid duplication of similar structures by determining the terminal residue number of each protein chain (e.g., polypeptide chain) from the temporary single chain after the correction, disconnecting a chemical bond angle potential at a border of the terminal residue, and adding an interatomic interaction potential at the border, and minimization (optimization) of an objective function representing a temporary energy value be performed by at least one of a simulated annealing method, a molecular dynamics calculation and a Monte Carlo method. As a result, construction and optimization of the Cα atomic coordinates are performed sequentially or simultaneously for all chains, thereby an objective model structure can be constructed.

For example, construction and optimization of coordinates of main-chain atoms of N, Cα, C and O (carbonyl oxygen) and coordinates of side-chains of amino acid residues can be performed sequentially or simultaneously for all chains by determining the number of the C terminal residue of each protein chain (e.g. polypeptide chain) from the sequence alignment corrected by the above-mentioned method when performing the potential calculation, disconnecting a chemical bond potential, a chemical bond angle potential and a chemical bond torsional angle potential at the boundary of the main-chain of the respective proteins and adding an interatomic interaction potential at the boundary. In addition, when using the modeling method, atomic coordinates of an amino acid residue of a protein-protein interaction region is determined accurately, and a protein-protein interaction model can be constructed.

In the above-mentioned method, it is preferable that in the case where at least two chains among the plural chains constituting the target protein are protein chains such as polypeptide chains, a data set of a modification whose similarity or the like is superior or inferior with the use of a potential energy value as an index be created based on a possible combination of 20 amino acids for each amino acid residue located at a mutual protein-protein recognition region, thereby enabling to construct a tertiary structure of the at least two protein chains whose functions as respective proteins are increased or decreased.

It is preferable that in the case where at least one chain among the plural chains constituting the target protein is an amino acid derivative, for example, a non-natural amino acid such as βAsp or γGlu or a derivative thereof, or a peptide derivative (a peptide ligand) (at least one chain of the plural chains is a polypeptide chain), and has a similar chemical structure to the corresponding ligand molecule in the reference protein, the alignment, in which the derivative of the target protein is defined as a new residue name and a one-character code and the ligand of the reference protein is defined as another new residue name and a one-character code, be created manually or automatically, and based on the possible combination of 20 amino acids and their derivatives for each residue constituting the ligand sequences, a rank be placed in the ascending order of the potential energy value, thereby enabling to construct a ligand model data set of the amino acid derivative or the peptide derivative, in which the several higher-ranked combinations are stored as modifications with superior similarity to a binding region of a receptor protein. By creating a data set in which the amino acid sequence of the ligand is mutated diversely, various peptide ligand models which can bind to the target protein can be constructed (refer to below-mentioned Example 3).

In the same way, it is preferable that in the case where the peptide ligand exists in the chain components of the plural chains constituting the target protein (at least one chain among the plural chains is a polypeptide chain), the amino acid sequence of the ligand be fixed, and based on the possible combination of 20 amino acids for each amino acid residue located at the region recognizing the ligand, a data set of a modification with superior similarity to a binding region of the plural higher-ranked receptor proteins be created with the use of the potential energy value as an index, thereby enabling to construct the tertiary structures of various ligand receptor proteins that can bind to the ligand. In this way, various receptor models that can bind to a specific ligand can be constructed.

In the above-mentioned method, the plural chains may be domains or modules into which a single chain polypeptide is divided, and can be restored to a single temporary chain. After assuming the protein as plural chains by dividing it into domains or modules, a highly accurate tertiary structure model can be attempted by restoring the protein to a temporary single chain with the use of the above-mentioned modeling method.

In the above-mentioned method, in the target protein (or the target sequence), a substance as a component of the plural chains (as a chain constituting the plural chains), which is neither a common amino acid nor a peptide, in which plural common amino acids are bound, and preferably, has been registered in the tertiary structure database (such as PDB) can be included. The examples include non-natural amino acids, pharmaceutical substances, nucleic acids, saccharides, organic compounds such as organic metal compounds, metal oxides and their ions, hetero components of inorganic compounds or the like such as metals and their ions. In the protein, even in the case where such a substance (e.g. as a ligand or the like) is included as at least one chain of the plural chains constituting the protein, one or more polypeptide chains are included to form the protein.

As the above-mentioned method, it can include steps of searching the reference protein which is appropriate for the target sequence from the tertiary structure database and performing the sequence alignment with the amino acid sequences of the searched plural reference proteins; selecting the amino acid sequence of the reference protein whose E-value is small for the target sequence; and adding the delimiter to the C terminal region (end) of the amino acid sequence of the chain included in the reference protein and also adding the delimiter to the corresponding position to the target sequence (correction of sequence alignment).

Addition of the delimiter as mentioned above is performed for binding all the plural chains one another to enable to handle the plural chains as a single chain. Therefore, it is not necessary to add the delimiter to the C terminal end of the last binding chain when making it a single chain (the last terminal end when making it a single chain). It is added in the same way, though it is not necessary, the added delimiter of the last terminal end can be disregarded.

In addition, a step of obtaining coordinates from the reference structure determined in the step of selecting the amino acid sequence of the reference protein for a Cα atom which is one of the constitutive atoms in an amino acid of the target sequence based on the alignment information as mentioned above and optimizing the Cα atomic coordinates used in the above-mentioned method of the invention (example: refer to claim 11); a step of adding main-chain atomic coordinates from the tertiary structure database to the obtained Cα atomic coordinates and optimizing the main-chain atomic coordinates used in the above-mentioned method of the invention; and adding side-chain atomic coordinates from the tertiary structure database to the obtained main-chain atomic coordinates and optimizing the side-chain atomic coordinates used in the above-mentioned method of the invention can be included.

As the above-mentioned potential formula, the potential formulae shown in Table 1 (mentioned below) can be adopted. The preferred are as follows.

In a potential formula when the total chain number=M, wherein N represents the number of protein chains such as polypeptide chains and k_{N} represents the serial number of the C terminal residue for the N-th protein chain (such as polypeptide chain) i=1, . . . , M−1 is simplistically described as i=1, M−1,

(A) in the calculation for construction and optimization steps of the Cα atomic coordinates, a case where i=k_{N(N=1,M-1)} of a temporary chemical bond potential is not included and cases where i=k_{N(N=1,M-1)}, i=k_{N(N=1,M-1)}+1 of a temporary chemical bond angle potential are not included. Also, in the case of an interatomic interaction potential, j>i+1 is added if i=k_{N}−1, and j>i is added if i=k_{N}.

(B) In the calculation for construction and optimization steps of the main-chain atomic coordinates, a bond between Ci and Ni+1 when i=k_{N(N=1,M-1)} is not included in a chemical bond potential, angles when i=k_{N(N=1,M-1}), Cαi-Ci-Nᵢ₊₁, Oi-Ci-Nᵢ₊₁ and Ci-Nᵢ₊₁-Cαᵢ₊₁ wherein C and O represent a carbon atom and an oxygen atom in a carbonyl respectively, Cα represents an α carbon atom and N represents a nitrogen atom are not included in a chemical bond angle potential, further, angles Ni-Cαi-Ci-Nᵢ₊₁, Cαi-Ci-Nᵢ₊₁-Cαᵢ₊₁, and Ci-Nᵢ₊₁-Cαᵢ₊₁-Cᵢ₊₁ when i=k_{N(N=1,M-1)} are not included in a chemical bond torsional potential. In addition, for an interatomic interaction potential, when the length between atoms is represented by r, a case of rᵢⱼ ≦ a specified value for rᵢⱼε{r_{Ni,Ni+1}; r_{Cαi,Ni+1}; r_{cαi,Cαi+1}; r_{Ci,Ni+1}; r_{Ci,Cαi+1}; r_{Ci,Cβi+1}; r_{Ci,Ci+1}; r_{Oi,Ni+1}; r_{Oi,Cαi+1}} when i=k_{N(N=1,M-1)} is added.

In other words, in the extended modeling method, when performing the potential calculation, by determining the C terminal residue number of each protein chain (such as polypeptide chain) from the sequence alignment corrected by the above-mentioned method, disconnecting the chemical bond potential, the chemical bond angle potential and the chemical bond torsional angle potential at the boundaries of the main-chains of the respective protein chains and adding the interatomic interaction potential at the boundaries, thereby construction and optimization of the coordinates of main-chain atoms of N, Cα, C and O (carbonyl oxygen atoms) and the coordinates of side-chains of the amino acid residues are performed sequentially or simultaneously for all chains, accordingly a model structure can be constructed.

FIG. 1 shows the relationship between the whole amino acid sequences of the protein and the serial number of the C terminal residues of each protein chain (such as polypeptide chain). The total chain number=M, each k_{N} is distinguished by the delimiter U. N represents the number of protein chains (such as polypeptide chains).

FIGS. 2 to 8 show specific descriptions of the correction method of the above-mentioned sequence alignment. As shown in FIGS. 2 to 4, disconnection of the chemical bond potential and the chemical bond angle potential between the C terminal residue k_{N} of the N-th protein chain and the N terminal residue k_{N}+1 of the (N+1)-th protein chain, and addition of the interatomic interaction potential are performed sequentially or simultaneously for all protein chains (such as polypeptide chains) from N=1 to (the total number of protein chains)−1 (M−1). In each step of construction and optimization of the main-chain atomic coordinates, as shown in FIGS. 5 to 8, disconnection of the chemical bond potential, the chemical bond angle potential, the chemical bond torsional angle potential between the C terminal residue k_{N} of the N-th protein chain and the N terminal residue k_{N}+1 of the (N+1)-th protein chain, and addition of the interatomic interaction potential are performed sequentially or simultaneously for all protein chains from N=1 to (the total number of protein chains)−1 (M−1). Thereby, not only appropriateness of the model structure can be improved, but also simplification and improvement of efficiency of the calculation steps can be realized.

Although there is no particular restriction on the input file format, for example, as a general formula for the input file format in the case where modeling of the target protein from the reference protein is performed, the following content can be adopted:

The target protein ID is written after > in the first line. The amino acid sequences of the target protein having the delimiter (string) added next to the terminal residue of each protein chain (such as polypeptide chain) are written for all the protein chains in the second line without line feed. The reference protein ID is written after > in the third line. Amino acid sequences of the reference protein having the delimiter (string) added next to the terminal residue of each protein chain (such as polypeptide chain) are written for all the protein chains in the fourth line without line feed. As a way to arrange the amino acid sequences in the second line and the fourth line, it is preferable that the positions of the same order of the delimiter (string) be made to always conform in the second line and the fourth line with the use of the alignment obtained as mentioned above.

This is illustrated as follows:
>(the target protein ID);
   (sequence of the first chain) (delimiter) (sequence of the second chain) (delimiter) . . . (delimiter) (sequence of the M-th chain);
>(the reference protein ID); (sequence of the first chain) (delimiter) (sequence of the second chain) (delimiter) . . . (delimiter) (sequence of the M-th chain).

Incidentally, the above brackets are used for easily viewable, therefore, it is preferable that a consecutive character string be made without brackets in practice.

In the method of the present invention, as mentioned above, the method of correcting the amino acid sequence alignment between the target protein composed of the plural chains and the reference protein to an alignment as a temporary single chain by inserting an identification mark at the boundary of each protein chain (such as polypeptide chain) is convenient and advantageous for performing the program. However, there is a method of converting plural chains to a temporary single chain other than this. For example, it is also feasible by a method of creating a separate file in which the boundary of each protein chain is registered sequentially with the residue number and sending it as a variable to a computer software program. Both are different only in the file format used for indicating the boundary of each protein chain, and the contents are exactly equivalent. In the same way, a method of using an identification number or the like as an internal variable indicating the boundary position or the boundary of each protein chain (such as polypeptide chain) obtained from the result of performing the sequence alignment procedure in the computer software program is also exactly equivalent. These methods can be, as a matter of course, used as the present invention.

The present invention is a method of constructing a tertiary structure of a protein (target protein) composed of plural chains having given arbitrary amino acid sequences by extending an comparative modeling method of constructing a tertiary structure of a protein composed of a single chain having a given arbitrary amino acid sequence (extended modeling method) Also, the present invention lies in a method of constructing a tertiary structure of a protein composed of plural chains characterized by assuming, for the target protein and the selected reference protein, each amino acid sequence of the plural chains, which are included in the target protein and the selected reference protein respectively and correspond each other, as a single chain in a state where the N terminal ends and the C terminal ends are bound sequentially, performing sequence alignment between the reference sequence of the thus obtained temporary single chain and the target sequence of the thus obtained temporary single chain to confirm their correspondence relationship, locating a Cα atom, which is one of the constitutive atoms in an amino acid residue in the target sequence, binding the Cα atoms by amide bonds, further adding side-chains to construct coordinates for other constitutive atoms, performing optimization, and constructing the tertiary structure by the modeling method.

As the selected reference protein, the one whose amino acid sequence of the protein chain, such as a polypeptide chain is similar, in particular, statistically significantly similar to the target protein can be preferably selected.

In the method of constructing a tertiary structure of the present invention, construction of the tertiary structure of the target protein can be performed by obtaining coordinates from the tertiary structure of the reference protein selected for Cα atoms in a main-chain amino acid in the target protein based on the obtained alignment information, optimizing the Cα atomic coordinates so as to minimize an objective function, adding coordinates (including Cβ atomic coordinates) of other atoms of a main-chain to the optimized Cα atomic coordinates, optimizing the main-chain atomic coordinates so as to minimize the objective function, adding coordinates of other atoms of side-chains to the optimized main-chain atomic coordinates, and optimizing the side-chain atomic coordinates so as to minimize the objective function.

The present invention, as another embodiment, also lies in a tertiary structure model of a protein characterized in that it is constructed by the above-mentioned method of the present invention, and further lies in a program (newFAMS) characterized in that it includes any of the above-mentioned methods of the invention or a computer characterized in that it is installed with the program.

The present invention, as another embodiment, lies in a database which can be used in an extended modeling method characterized in that a data composed of the tertiary structure model of the protein constructed by the above-mentioned method of the present invention, the ligand model used in the above-mentioned method of the present invention and the tertiary structure model of the ligand receptor protein used in the same way is fixed and combined.

The present invention, as another embodiment, lies in a database characterized in that it is constructed from a data of the tertiary structure model of the protein constructed by the above-mentioned method of the present invention, the ligand model used in the above-mentioned method of the present invention and the tertiary structure model of the ligand receptor protein used in the same way so as to enable to browse or search the data by a computer.

The present invention, as another embodiment, lies in a database structure characterized in that the following content can be browsed or searched by a computer:
a gene identification code or a protein identification code of the target protein composed of plural chains, an about one-line function description, the target amino acid sequence and (coordinates of) three-dimensional tertiary structure of the target protein;
a gene identification code or a protein identification code of the reference protein, an about one-line function description, the reference amino acid sequence and a (coordinate of) three-dimensional tertiary structure of the reference protein; and
an alignment result between the target sequence and the reference sequence, a homology value and an E-value.

The present invention, as another embodiment, lies in a computer software program characterized in that any of the above-mentioned content of the database can be browsed or searched, or the database structure can be used, or a computer installed with the program.

The present invention further lies in an interface characterized in that it is designed to enable to access an target protein, preferably to enable to simply and easily access an target protein without a preliminary knowledge, by conjunction search such as partial agreement of an arbitrary symbol specific to living species, a protein code name, a reference protein name, a character string of an about one-line function description for a protein, which is desired to be browsed, among the tertiary structure database constructed by any of the above-mentioned method of the present invention.

By the above-mentioned method, the atomic coordinates specifying the tertiary structure of the protein having the plural chains can be provided. In this description, Cα atom means a carbon atom, which is the center of the backbone of each amino acid residue. The Cα atom of each amino acid residue except for glycine has a property of optical activity. Cβ atom means a carbon atom, which binds to a side-chain side of the Cα atom. C atom means a carbon atom of a carbonyl group, which binds to the Cα atom.

In Table 1, N represents the number of protein chains such as polypeptide chains, k_{N} represents the serial number of the C terminal residue in the N-th protein chain (such as polypeptide chain), and when the total chain number=M, i=1, . . . , M−1 is represented by i=1, M−1. FIG. 1 shows the relationship between the whole amino acid sequences of the protein and k_{N}. For each potential formula in Table 1, specific illustrations for handling each protein chain (such as polypeptide chain) at the boundary are shown in FIGS. 2 to 8, and will be detailed in the following paragraph.

[Table 1]

Calculation Conditions of Potential Function at Boundary of Each Protein Chain

It is based on a conventional method (refer to K. Ogata and H. Umeyama, Journal of Molecular Graphics and Modeling 18, 258-272, 2000, or the like). In the method, a newly revised part in the extended modeling method for a protein of plural chains of the present invention with the content, which is not known nor suggested in a conventional single-chain modeling, is shown by “provided that:” (provisio) in the below-mentioned potential formulae. In other words, the conventional modeling method is used for apart other than the below-mentioned provisio. Therefore, the content of the provisio corresponds to the modified content, which can be newly adopted for a temporary single chain modeling used in the present invention. Unless otherwise explicitly stated, the meanings of the characters in the formulae all accord with those in the above-mentioned literature of the conventional method. The constants in the formulae can vary within a reasonable and appropriate range, and are not limited to the below-mentioned specific values. Here, the whole content of the literature of the conventional method is included for reference in this description as a part.

(A) Construction and Optimization Steps of Cα Atomic Coordinates

Calculation Condition of Chemical Bond Potential

$${E}_{len} = {K}_{l} {\sum }_{i} {( {D}_{i , i + 1} - 3.8 )}^{2}$$

Provided that: i≠k_{N(N=1,M-1)}
Calculation Condition of Chemical Bond Angle Potential

$${E}_{ang} = {K}_{a} {\sum }_{i} {( {θ}_{i} - {θ}_{0} )}^{2}$$

Provided that: i≠k_{N(N=1,M-1)}, i≠k_{N(N=1,M-1)}+1
Calculation Condition of Interatomic Interaction Potential

$${E}_{vdw} = {K}_{vdw} {\sum }_{i , j ( > i + 2 )} \{ {( \frac{3.8}{{D}_{i , j}} )}^{12} - 2 {( \frac{3.8}{{D}_{i , j}} )}^{6} \}$$

Provided that: j>i+1 if i=k_{N}−1, j>i if i=k_{N}
Potential function in which calculation conditions of the conventional method can be used:
Calculation Condition of S—S Bond Potential

$${E}_{ss} = {K}_{ss} {\sum }_{i} {( {D}_{i}^{SS} - 5.4 )}^{2}$$

Calculation Condition of Coordinate Positional Potential

$${E}_{pos} = {K}_{pos} {\sum }_{i} \frac{1}{{M}_{i}} {‖ {x}_{i} - < {w}_{i} {x}_{i} > ‖}^{2}$$

(B) Construction and Optimization Steps of Main-Chain Atomic Coordinates
Calculation Condition of Chemical Bond Potential

$${E}_{bond} = {K}_{b} {\sum }_{i} {( {b}_{i} - {b}_{i}^{0} )}^{2}$$

Provided that: if i=k_{N(N=1,M-1)} and bᵢ=D_{Ci,Ni+1}, bᵢ−bᵢ⁰=0
Calculation Condition of Chemical Bond Angle Potential

$${E}_{ang} = {K}_{a} {\sum }_{i} {( {θ}_{i} - {θ}_{i}^{0} )}^{2}$$

Provided that:
if i=k_{N(N=1,M-1)} and θᵢ=θ_{Cαi,Ci,Ni+1}, θᵢ−θᵢ⁰=0
If i=k_{N(N=1,M-1)} and θᵢ=θ_{Oi,Ci,Ni+1}, θᵢ−θᵢ⁰=0
if i=k_{N(N=1,M-1)} and θᵢ=θ_{Ci,Ni+1,Cαi+1}, θᵢ−θᵢ⁰=0
Calculation Condition of Chemical Bond Torsional Angle Potential

$${E}_{tor} = {K}_{t} {\sum }_{i} \sqrt{{( {ϕ}_{i} - {ϕ}_{i}^{0} )}^{2} + {( {ψ}_{i} - {ψ}_{i}^{0} )}^{2}} + {K}_{ω} {\sum }_{i} {( {ω}_{i} - {ω}_{i}^{0} )}^{2}$$

Provided that: if i=k_{N(N=1,M-1)}, φᵢ−φᵢ⁰=ωᵢ−ωᵢ⁰=φᵢ₊₁−φᵢ₊₁=0
Calculation Condition of Interatomic Interaction Potential

$${E}_{non \text{-} bond} = {K}_{non} {\sum }_{i , j} {ɛ}_{i , j}^{*} \{ {( \frac{{r}_{i , j}^{*}}{{r}_{i , j}} )}^{12} - 2 {( \frac{{r}_{i , j}^{*}}{{r}_{i , j}} )}^{6} \}$$

Provided that: when i=k_{N(N=1,M-1)}, if rᵢⱼ≦8.0 for rᵢⱼε{r_{Ni,Ni+1}; r_{Cαi,Ni+1}; r_{Cαi,Cαi+1}; r_{Ci,Ni+1}; r_{Ci,Cαi+1}; r_{Ci,Cβi+1}; r_{Ci,Ci+1}; r_{Oi,Ni+1}; r_{Oi,Cαi+1}}, the case is included in the calculation of E_{non-bond}.
Potential function in which calculation conditions of the conventional method can be used:
Calculation Condition of S—S Bond Potential

$${E}_{SS} = {\sum }_{i} \{ {{K}_{C α}^{SS} ( {D}_{i}^{C α} - 5.4 )}^{2} + {{K}_{C β}^{SS} ( {D}_{i}^{C β} - 3.8 )}^{2} \}$$

Calculation Condition of Coordinate Positional Potential

$${E}_{pos} = {K}_{pos} {\sum }_{i} \frac{1}{{M}_{i}} {‖ {x}_{i} - 〈 {w}_{i} {x}_{i} 〉 ‖}^{2}$$

Calculation Condition of Potential with Respect to Amino Acid Residue Chirality

$${E}_{chi} = {K}_{chi} {\sum }_{i} {( {τ}_{i} - \frac{2}{3} π )}^{2}$$

Calculation Condition of Hydrogen Bond Potential Between Main-Chains

$${E}_{hydr} = {K}_{hydr} {\sum }_{i , j} {( {D}_{i , j}^{N - O} - 2.9 )}^{2}$$

As mentioned above, in the method of the present invention, by correcting the input file format of the plural chains and the potential formulae so as to present a morphology as a temporary single chain respectively, the input file format and the interaction between protein chains (such as polypeptide chains), which generally become more complicated with the increase in the number of chains, can be always described with the simplest input format and the clearest potential formula.

### BRIEF DESCRIPTION OF THE DRAWINGS

[FIG. 1]

FIG. 1 shows the relationship between the whole amino acid sequences of a protein used for explaining the present invention and the serial number k_{N} of the C terminal residues of each chain.

The total chain number=M, each k_{N} is distinguished by a delimiter U. N represents the number of protein chains.

[FIG. 2]

FIG. 2 illustrates how to handle C and N terminals in the Elen term of the potential formula (limited to the case of Cα).

[FIG. 3]

FIG. 3 illustrates how to handle C and N terminals in the Eang term of the potential formula (limited to the case of Cα).

[FIG. 4]

FIG. 4 illustrates how to handle C and N terminals in the Evdw term of the potential formula (limited to the case of Cα).

[FIG. 5]

FIG. 5 illustrates how to handle C and N terminals in the Ebond term of the potential formula.

[FIG. 6]

FIG. 6 illustrates how to handle C and N terminals in the Eang term of the potential formula.

[FIG. 7]

FIG. 7 illustrates how to handle C and N terminals in the Etor term of the potential formula.

[FIG. 8]

FIG. 8 illustrates how to handle C and N terminals in the Enon-bond term of the potential formula.

[FIG. 9]

FIG. 9 is a flow chart showing an example of a method of constructing a tertiary structure of a protein composed of plural chains according to the present invention.

[FIG. 10]

FIG. 10 illustrates an example of a method of constructing Cα atomic coordinates of the target protein.

The matched portion of an alignment is obtained from the reference protein, and for the non-matched portion, a region having the least rmsd by superposition of two residues of N and C terminals is obtained from a database (refer to K. Ogata and H. Umeyama, Journal of Molecular Graphics and Modeling 18, 258-272, 2000).

[FIG. 11]

FIG. 11 illustrates the concept of local space homology (LSH).

For example, in the calculation with respect to the central T residues within the circle (sphere) of the figure, they are considered by the gray colored residues. The region enclosed by rectangles in the alignment is the residue pairs to be considered and the ratio of residues marked with asterisks is LSH (56.2% in this example) (refer to K. Ogata and H. Umeyama, Journal of Molecular Graphics and Modeling 18, 258-272, 2000).

[FIG. 12]

FIG. 12 illustrates the relationship between the local space homology (LSH) and the ratio contained in structurally conserved regions (SCRs).

LSH is calculated from the superposition of Cα atoms in the target protein and the reference protein, and the ratio contained in SCRs indicates the number of residues in SCRs for the total number of residues in the target protein (refer to K. Ogata and H. Umeyama, Journal of Molecular Graphics and Modeling 18, 258-272, 2000).

[FIG. 13]

FIG. 13 shows the whole amino acid sequences of metabotropic glutamate receptor 1 derived from rat used in Example 1.

[FIG. 14]

FIG. 14 is composed of FIG. 14-1 and FIG. 14-2 and shows the alignment result of MGR1_RAT and MGR5_RAT in Example 1.

[FIG. 15]

FIG. 15 shows the alignment result of MGR1_RAT and MGR5_RAT in Example 1.

Only portion, in which coordinates exist in 1EWT, is shown.

[FIG. 16]

FIG. 16 shows the input file format for the conventional FAMS in Example 1.

[FIG. 17]

FIG. 17 shows a monomer model of mGlu receptor Swiss Prot [MGR5_RAT] in Example 1.

[FIG. 18]

FIG. 18 shows the input file format for newFAMS in Example 1.

[FIG. 19]

FIG. 19 shows a dimer model of mGlu receptor Swiss Prot [MGR5_RAT] in Example 1.

[FIG. 20]

FIG. 20 shows the enlarged drawing of the bound surface of the dimer model of mGlu receptor Swiss Prot [MGR5_RAT] in Example 1.

[FIG. 21]

FIG. 21 shows the amino acid sequences of 1JSQ monomer in Example 2.

[FIG. 22]

FIG. 22 shows the alignment result of [O93437] and MSBA in Example 2.

[FIG. 23]

FIG. 23 shows the new alignment result of [O93437] and MSBA in Example 2.

[FIG. 24]

FIG. 24 shows the input file format of newFAMS for [O93437] in Example 2.

[FIG. 25]

FIG. 25 shows a monomer model of ABC transporter SwissProt [O93437] in Example 2.

[FIG. 26]

FIG. 26 shows the input file format for a homodimer used in newFAMS in Example 2.

[FIG. 27]

FIG. 27 shows a dimer model of ABC transporter SwissProt [O93437] in Example 2.

[FIG. 28]

FIG. 28 illustrates the bound surface of the dimer model of ABC transporter SwissProt [O93437] in Example 2.

[FIG. 29]

FIG. 29 illustrates the hydrophobic region of the ABC transporter model in Example 2.

[FIG. 30]

FIG. 30 shows the molecular structure of estradiol in Example 2.

[FIG. 31]

FIG. 31 shows the amino acid sequences of PDB: 1JKY in Example 3.

PDB: 1JKY contains A chain (LF) and B chain (16 residues of MAPKK).

[FIG. 32]

FIG. 32 illustrates MAPKK-2 in LF in Example 3.

In addition, ball and stick models are shown for Leu-2 and Ala-3.

[FIG. 33]

FIG. 33 shows the input file format of newFAMS in the peptide modification from [LA] to [FF] in Example 3.

[FIG. 34]

FIG. 34 illustrates a MAPKK-2 modification form in LF in Example 3.

In addition, ball and stick models are shown for Phe-2 and Phe-3.

[FIG. 35]

FIG. 35 shows the amino acid sequences of H chain and P chain of 1BTH in Example 4.

[FIG. 36]

FIG. 36 shows the amino acid sequences of E chain and I chain of 2PTC in Example 4.

[FIG. 37]

FIG. 37 illustrates the alignment of H chain of 1BTH and E chain of 2PTC in Example 4.

[FIG. 38]

FIG. 38 illustrates the alignment of P chain of 1BTH and I chain of 2PTC in Example 4.

[FIG. 39]

FIG. 39 shows the input file format of the conventional FAMS in Example 4.

[FIG. 40]

FIG. 40 shows the input file format of newFAMS in Example 4.

[FIG. 41]

FIG. 41 illustrates the bound surface of the model of H chain and P chain of 1BTH in Example 4.

[FIG. 42]

FIG. 42 shows the superposed tertiary structures of the 1BTH (H chain and P chain) model, which is modeled from 2PTC and the 1BTH (H chain and P chain) model, which is actually registered in PDB.

The darker one was obtained by actual X-ray crystallographic analyses and the lighter one was obtained by performing the modeling this time.

[FIG. 43]

FIG. 43 illustrates an example of interface screens to a tertiary structure database in Example 5.

It is designed that a list of models whose three-dimensional coordinates can be browsed is displayed and an alignment is displayed by clicking the right button thereby three-dimensional coordinates can be obtained.

[FIG. 44]

FIG. 44 conceptually illustrates and compares the case of modeling a tertiary structure by the present invention with the case of modeling by the conventional method when selecting a tertiary structure in which domains (plural chains) separated in the sequence or separated widely are contacted in the space as a reference protein.

FIG. 44a: The result of the tertiary structure of a complex in the state where domains (plural chains) separated in the sequence or separated widely are contacted in the space, is experimentally determined; FIG. 44b: The result of modeling by the conventional method in which the structure shown in FIG. 44a was used as a reference protein (a not-preferred model); FIG. 44c: The result of modeling by the present invention in which the structure shown in FIG. 44a was used as a reference protein (a preferred good model).

### EMBODIMENTS OF THE INVENTION

Hereinafter, embodiments of the present invention will be described. The present invention will be detailed mainly describing a homology modeling method as a preferred and typical example. However, these are only for the purpose of illustrating typical examples, therefore the present invention is not limited thereto.

The present invention expands the applicable range of the protein Full Automatic Modeling System (FAMS: refer to K. Ogata and H. Umeyama, Journal of Molecular Graphics and Modeling 18, 258-272, 2000. The whole content is included for reference in this description as a part) developed in the Department of Biomolecular Design (Professor Hideaki Umeyama), School of Pharmaceutical Sciences, Kitasato University, and improves the system.

Several terms are used in this description and unless otherwise explicitly stated, their definitions are as follows.

“Target protein” means a protein whose complete tertiary structure has not been determined by an X-ray crystallographic analysis, an NMR analysis, or the like and which is the subject of tertiary structure construction in the present invention. The amino acid sequence of this protein may be referred to as “target sequence” or “target amino acid sequence” in some cases. This target protein may include a protein whose partial structure has been determined but complete tertiary structure has not been determined, a protein whose function has been specified, a protein whose function has been presumed, a protein whose amino acid sequence has been determined but function has not at all been known, and the like. “Reference protein” means a protein whose detail tertiary structure has been already determined by an X-ray crystallographic analysis or an NMR analysis, and which is referred to for alignment or optimization of atomic coordinates. The amino acid sequence of this protein may be referred to as “a reference sequence” or “a reference amino acid sequence” in some cases.

“Alignment” means, in the case where amino acid sequences of at least two types of proteins are matched, to mutually correlate them, and the method is detailed in the following explanation of each step.

“Atomic coordinates” describes a tertiary structure in three-dimensional coordinates. It is a relative distance in three directions perpendicular to each other with a certain point in the space as the origin, and is a vector quantity composed of three numbers per atom excluding hydrogen atoms existing in a protein.

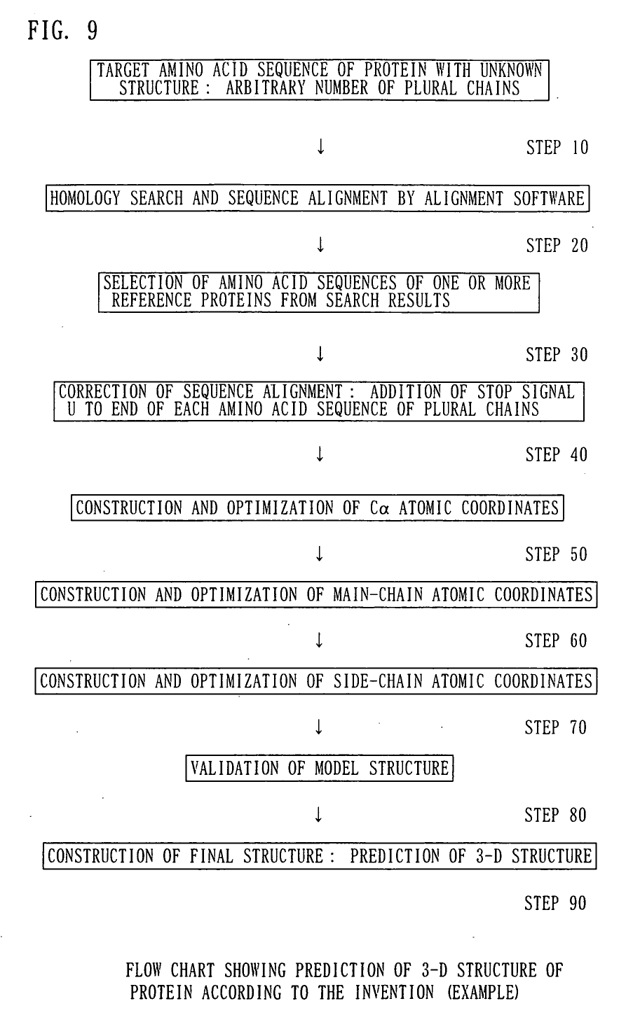

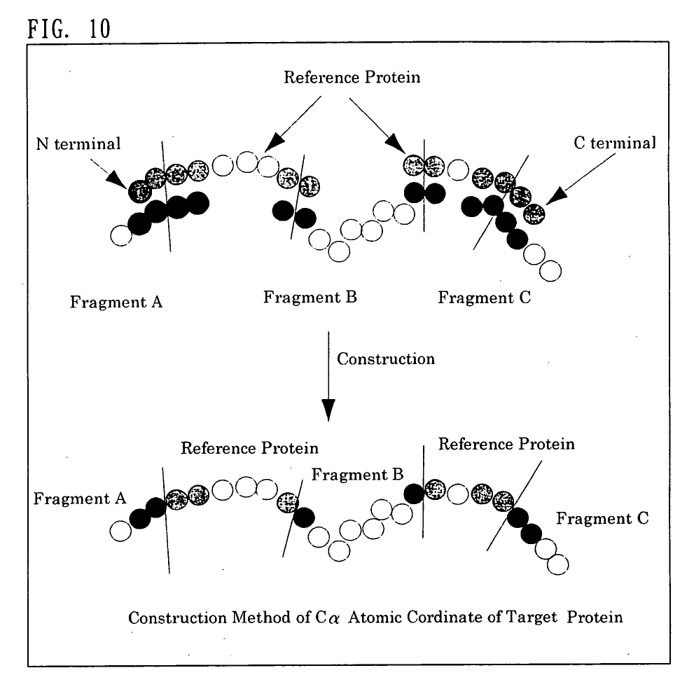

FIG. 9 is a flow chart showing an example of the method of constructing a tertiary structure of a protein composed of plural chains according to the present invention.

As shown in FIG. 9, in this method (an example), first an amino acid sequence of a protein with an unknown tertiary structure (hereinafter referred to as “target sequence”) is prepared in Step 10. In Step 20, a reference protein (a reference amino acid sequence) is selected from a tertiary structure database by using FASTA, PSI-BLAST or LIBRA. Using 20 types of characters, which represent amino acid residues, as an index, the alignment of the target sequence and the selected reference amino acid sequence is performed. In Step 30, amino acid sequences of one or more reference proteins are selected based on the search results. In Step 40, the character “U” is added to the C terminal region (end) of each amino acid sequence of the plural chains as a delimiter, and the sequence alignment is corrected by similarly adding “U” as a delimiter sequentially to the end of each amino acid sequence at the position corresponding to the target sequence. At this time, it is not necessary to add the above-mentioned delimiter to the terminal region of a chain corresponding to the last chain (the terminal end of a chain constituting a temporary single chain; the last terminal end of a temporary single chain) as mentioned above. Even if it is added, the delimiter added to the last terminal end can be disregarded. In Step 50, based on the alignment information, coordinates are obtained for Cα atoms, which are one of the constitutive atoms of an amino acid residue, from the determined reference structure in Step 30, and the Cα atomic coordinates are optimized so as to minimize the objective function (E_{Cα}) composed of the sum of various potential terms mentioned below by a simulated annealing method. In Step 60, main-chain atomic coordinates are added to the Cα coordinates obtained in Step 50 from a database, and the main-chain atomic coordinates are optimized so as to minimize the objective function (Eₘₐᵢₙ) composed of the sum of various potential terms mentioned below by the simulated annealing method. In Step 70, side-chain atomic coordinates are added to the main-chain atomic coordinates obtained in Step 60 from a database, and a tertiary structure is constructed by the same simulated annealing method as in Step 60. In Step 80, varidity of the tertiary structure of the completed model is evaluated as mentioned below and the final structure is attained in Step 90. The validity of the model structure is evaluated by superposing the main-chain atomic coordinates of the model and the reference protein for regions excluding a loop insertion or a deletion region, and the model is judged appropriate (valid) if rmsd is, for example, 1 Å or less.

The present invention has been developed, differing from the conventional FAMS, by correcting Steps 50, 60 and 70, which could only perform the structure optimization with the use of a reference structure of a single chain conventionally, thereby enabling to apply to also plural chains. Hereinafter, each step is further detailed as a preferred embodiment.

Step 10: Amino Acid Sequence of a Protein with Unknown Structure

First, an amino acid sequence of a target protein with an unknown structure (target sequence) is prepared. As an amino acid sequence of a used target protein, a sequence of any origin, such as one registered in a database or one analyzed for the sequence for the first time, may be used. In addition, a sequence of a protein whose partial structure only has been analyzed may also be the subject of construction of a tertiary structure in the present invention to obtain the information of complete tertiary structure. The database, which can be used, includes, for example, human (H. sapiens), drophila (D. melanogaster) nematode (C. elagans), yeast (S. cerevisiae), Arabidopsis thaliana (A. thaliana) and the like registered in a database such as GenBank: ftp://ncbi.nlm.nih.gov/genbank/genomes/, PIR: http://www-nbrf.georgetown.edu/pir/ (National Biomedical Research Foundation (NBRF)), Swiss Plot: http://www.expasy.ch/sprot/sprot-top.html (Swiss Institute of Bioinformatics (SIB), European Bioinfomatics Institute (EBI)), TrEMBL (the same URL and administrator as Swiss Plot), TrEMBLNEW (the same URL and administrator as Swiss Plot) and DAD: ftp://ftp.ddbj.nig.ac.jp (Japan DNA Data Bank).

These databases are merely examples and any databases may be used as long as they are registered with amino acid sequences of proteins.

Step 20: Database Search and Sequence Alignment by Alignment Software such as FASTA, PSI-BLAST or LIBRA

Preferable softwares for the alignment of amino acid sequences prepared in Step 10 are, for example, FASTA, PSI-BLAST (Position-Specific Iterated BLSAT), LIBRA and the like.

FASTA is a program that searches a highly homologous sequence with a target sequence from a tertiary structure database and calculates the final similarity of the target sequence and the reference protein as an E-value. The details of FASTA are described in “Effective protein sequence comparison” Pearson W R, (1996) Methods Enzymol; 266: 227-58.

PSI-BLAST is programmed so as to perform profile alignment. The details of PSI-BLSAT are described in “Matching a protein sequence against a collection of PSI-BLAST-constructed position-specific scorematrices” Schaffer A A, Wolf Y I, Ponting C P, Koonin E V, Aravind L and Altschul S F, Bioinformatics 1999, 12, 1000-11.

PSI-BLSAT, which performs profile alignment, is a tool provided with the highest performance at present in the search of similarity of sequences. PSI-BLAST is one of the series of programs that search similar proteins called BLAST and output alignments. As the programs showing equivalent performance recently, RPS-BLAST and IMPALA are available (refer to A. A. Schaffe et al., Bioinformatics, 15 (12), 1000-1011, 1999). This program draws profile information only from statistically significant alignment relationship in the databases and generates a position specific score matrix (a matrix indicating a probability of substitution of each residue in the amino acid sequences with a certain amino acid residue statistically) of an amino acid sequence. Then inside the program, sequences having high similarity with the generated position specific score matrix instead of the target protein sequence are searched from a database, and the position specific score matrix is serially revised each time until significant alignments are not searched when the smallness of E-value is regarded as the limit. Then, the similarity of the final position specific score matrix and the reference protein is calculated as an E-value.

“E-value” quantitatively describes random background noise that exists for matches between sequences. It indicates how well two sequences are matched and it has a property of decreasing exponentially against scores representing sequence similarity and it is useful as a method for setting a threshold value when evaluating results. In PSI-BLAST, it corresponds to the case where the size of E-value has a value of normally 0.1 or less, preferably 0.001 or less (refer to A. A. Schaffe et al., Bioinformatics, 15 (12), 1000-1011, 1999).

Homology search is performed for the reference protein sequence from the tertiary structure database PDB and the sequence alignment of the searched reference sequence and the target sequence is performed using such a program.

Here, “reference protein” is a data of a sequence and a three-dimensional atomic coordinates obtained from the tertiary structure database. It can be obtained from public databases registered as the protein data bank (PDB).

In PDB database, 26243 tertiary structures were registered as of November 2001. As an example, ones having sequence similarity of 95% or more are judged to be in the same category and the longest sequence in each category, and the structure having the highest X-ray resolution if the size is the same, is selected as the representative of the category. A tertiary structure database used in the present invention is a database, in which these representatives have been collected. The representative structures of 3922 were used as the PDB database when the present invention was performed.

Step 30: Selection of Amino Acid Sequences of One or More Reference Proteins Based on the Search Results

Based on the results of the homology search, amino acid sequences of one or more reference proteins that have statistically significant similarity to the target sequence are selected.

Step 40: Correction of Sequence Alignment by Inserting Delimiter U at the End of Each Amino Acid Sequence of Plural Chains

In the case where the target sequence of the protein composed of plural chains was aligned with each reference sequence of the plural proteins in Step 30, modeling was performed for each chain of the target sequences conventionally. However, in the present invention, a delimiter (e.g. the character “U”) is inserted in the C terminal region (end) of each amino acid sequence of the plural chains, and alignment for performing simultaneously the modeling of all the target protein chains (such as polypeptide chains) as a temporary single chain is prepared. The higher limit of the number of protein chains, which can be calculated, was actually set to preferably 36 by referring to the proteins of plural chains registered in PDB, however it can be theoretically expanded to the number necessary for modeling or to the capacity limit of the computer to be used.

Step 50: Construction of Initial Cα Atomic Coordinates

Using the alignment including the delimiter in Step 40, based on the comparison of the target sequence with the reference sequences, information is obtained for amino acid residues that have an insertion or a deletion. A region in which three or more consecutive amino acid residues are matched in the alignment is selected, and in the region, the same one as the reference proteins is applied to as the Cα atom of the target protein for these pairwise amino acid residues. In the case where a Cα atom could not be obtained, coordinates are applied to from the peptide fragment database (refer to K. Ogata and H. Umeyama, Journal of Molecular Graphics and Modeling 18, 258-272, 2000) composed of Cα atoms constructed beforehand from PDB (refer to FIG. 10).

Step 50 (1): Optimization of Cα Atom by Simulated Annealing Method

The Cα atom constructed in the above Step 50 is optimized using the objective function (E_{Cα}) obtained by referring to the reference protein coordinates using the simulated annealing step explained in the paragraph of the above-mentioned background art. This objective function is as the below-mentioned formula (1). One of the important differences between the conventional method and the present invention is that the chemical bond potential Eₗₑₙ, the chemical bond angle potential E_{ang} and the interatomic interaction potential E_{vdw} in the formula (1) are corrected as below with the k_{N} value (equal to the serial number of the C terminal residue of the N-th protein chain) determined by referring to the delimiter U in the alignment created in Step 40.
E_{Cα}=Eₗₑₙ+E_{ang}+E_{vdw}+Eₛₛ+Eₚₒₛ (1)

Eₗₑₙ relates to the distance between Cα atoms of the residues located side by side in the sequence and is defined by the formula (2) below.

$${E}_{len} = {K}_{i} {\sum }_{i} {( {D}_{i , i + 1} - 3.8 )}^{2} \\ ( i \neq {k}_{N ( N = 1 ; M - 1 )} ) \qquad ( 2 )$$

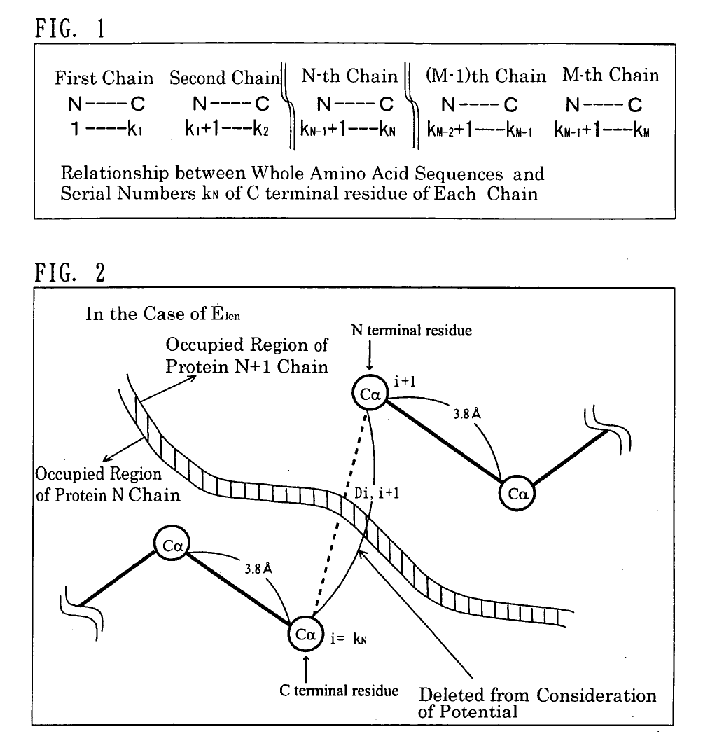

Here, D_{i,i+1} is the distance between Cα atoms of the residue i and the residue i+1. K₁ is a constant and, for example, set to 2. However, a chemical bond does not exist between the C terminal residue k₁ of the first protein chain and the N terminal residue k₁+1 of the second protein chain, therefore the case of i=k₁ is not included in the calculation of Eₗₑₙ. In the same way, as shown in FIG. 2, a chemical bond does not exist between the C terminal residue k_{N} of the N-th protein chain and the N terminal residue k_{N}+1 of the (N+1)-th protein chain, therefore the case of i=k_{N} is not included in the calculation of Eₗₑₙ (hereinafter, such a procedure is referred to as “disconnection of interaction”). In the case where the total number of protein chains is M, this procedure is performed from N=1 to M−1, and the residue numbers from k₁ to k_{M-1} can be specified by the positions of U from the first delimiter U₁ to the (M−1)-th delimiter U_{M-1} in the alignment created in Step 40.

Next, E_{ang} is a function of the chemical bond angle of Cα atoms and is as the formula (3) below:

$${E}_{ang} = {K}_{a} {\sum }_{i} {( {θ}_{i} - {θ}_{0} )}^{2} \qquad ( 3 )$$

provided that i≠k_{N(N=1,M-1)}, i≠k_{N(N=1,M-1)}+1.

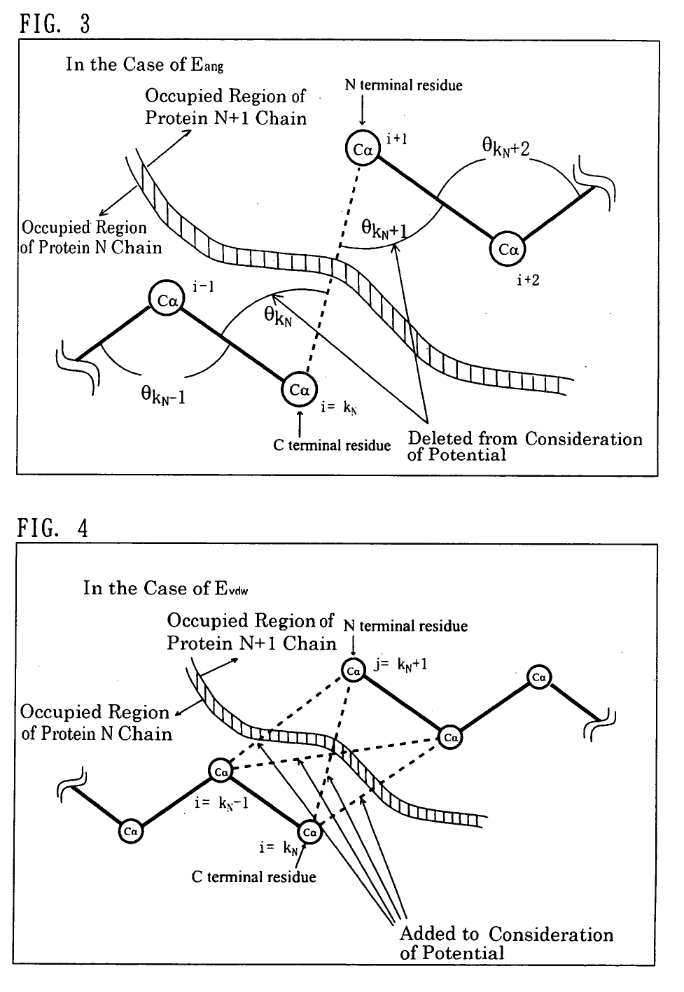

Here, θᵢ(rad) is the angle of Cα atoms of the i-th, (i+1)-th and (i+2)-th residues. θ₀ is defined as (100/180)π(rad) based on the X-ray structure of PDB. Kₐ is a constant and, for example, set to 1. However, regarding the bond angle potential E_{ang}, as shown in FIG. 3, disconnection of interaction procedure is performed in the same manner as Eₗₑₙ. In other words, the cases of i=k_{N} and i=k_{N}+1 are not included in the calculation of E_{ang}. This disconnection of interaction procedure is performed in the amino acid residues from k₁ to k_{M-1}.

Next, E_{vdw} is the van der Waals potential between Cα atoms and generally considered for residues separated by three or more residues and is as the formula (4) below:

$${E}_{vdw} = {K}_{vdw} {\sum }_{i , j ( > i + 2 )} \{ {( \frac{3.8}{{D}_{i , j}} )}^{12} - 2 {( \frac{3.8}{{D}_{i , j}} )}^{6} \} \qquad ( 4 )$$

provided that j>i+1 if i=k_{N}−1 and j>i if i=k_{N}.

Here, D_{i,j} is the distance between the pairwise atoms i,j that are within 6 Å0 from the i-th Cα atom, which is the subject, and the value of K_{vdw} is set to 0.01 (D_{i,j}≦3.2 Å) or 0.001 (D_{i,j}>3.2 Å). However, as shown in FIG. 4, because a chemical bond does not exist between the C terminal residue k_{N} of the N-th protein chain and the N terminal residue k_{N}+1 of the N+1-th protein chain, the calculation of E_{vdw} must be performed under the conditions that j>i+1 if i=k_{N}−1 and j>i if i=k_{N}. In the case where the total number of protein chains is M, this procedure is performed from N=1 to M−1, and the residue numbers from k₁ to k_{M-1} can be specified by the positions of the delimiter U from U₁ to U_{M-1} in the alignment created in Step 40.

Next, Eₛₛ relates to the distance between the Cα atoms of Cys residues which make a pair and forms an S—S bond and is defined by the formula (5) below.

$${E}_{ss} = {K}_{ss} {\sum }_{i} {( {D}_{i}^{SS} - 5.4 )}^{2} \qquad ( 5 )$$

Here, Dᵢ^{ss} is the distance between the pairwise Cα atoms of Cys residues that form a disulfide bond within a protein chain or between protein chains. Because the serial numbers throughout all the protein chains are used for the residue number i in the present invention, Eₛₛ between protein chains can be handled with the conventional potential function. Kₛₛ is a constant and set to, for example, 5.

Next, Eₚₒₛ is a function related to the position of Cα atoms and defined by the formula (6) below. This energy term is introduced for the purpose of maintaining the positions of Cα atoms relatively stable in the protein SCRs (Structural Conserved Regions: mentioned below).

$${E}_{pos} = {K}_{pos} {\sum }_{i} \frac{1}{{M}_{i}} ∥ {x}_{i} - < {w}_{i} {x}_{i} > {∥}^{2} \qquad ( 6 )$$

Here, Xᵢ represents the coordinate of the i-th Cα atom and Mᵢ represents the average distance between Cα atoms that are structurally equivalent on the alignment based on the structure or, in other words, located most closely in the three-dimensional coordinate system. When Mᵢ value cannot be obtained for the residue i or, in other words, a certain amino acid residue of the target sequence cannot be correlated with the Cα of the reference sequences, Mᵢ value is set to 10. |•| means a norm (distance between coordinate vectors), and <wᵢxᵢ> means the average coordinate of Cα atoms and is as the formula (7) below:

$$< {w}_{i} {x}_{i} >= \frac{1}{W} {\sum }_{j} {w}_{i}^{j} {x}_{i}^{j} \qquad ( 7 )$$

provided that j≠i.

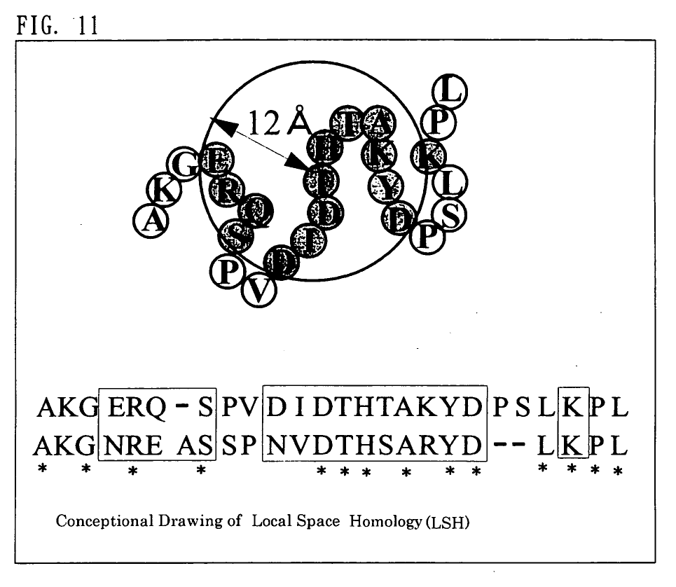

Here, x^{j}ᵢ represents a Cα atomic coordinates corresponding to the i-th residue of the j-th reference protein and w^{j}ᵢ represents a weight for the position of the i-th Cα atom of the j-th reference protein and W is the sum of w^{j}ᵢ for j. This w^{j}ᵢ is an important parameter because it determines a rough frame of the target protein. It is determined by a local value of spatial proximity within 12 Å of a given region called local space homology (LSH) (refer to K. Ogata and H. Umeyama, Journal of Molecular Graphics and Modeling 18, 258-272, 2000) as shown in FIG. 11. The relationship between LSH and the ratio of the pairwise residues belonging to the regions where structures are well conserved (SCRs: Structural Conserved Regions) is very high as shown in FIG. 12. This means that the position of the Cα atom is statistically located within 1.0 Å compared with the reference proteins when it has a high LSH value.

Cα atoms are optimized repeatedly according to the formula (1) using the simulated annealing method. In this optimization step, the perturbation of Cα atoms is set, for example, within 1.0 Å. In addition, this annealing step is repeated, for example, 100 times for all Cα atoms respectively. The parameter corresponding to the temperature is started at 25 and decreased by a factor of 0.5 at every step until 0.01 is reached and then using a constant value for the parameter.

From the result of the superposition of the tertiary structures, the acquisition of structural information from the reference sequence having the least number of insertion and deletion to the target sequence and the construction of the Cα atoms are repeated ten times, and the Cα atomic coordinates with the least value of the objective function is calculated as an optimal solution.

Step 60: Construction and Optimization of Main-Chain Atomic Coordinates

Coordinates of other main-chain atoms (an N atom of amide, a C atom of carbonyl and an O atom of carbonyl) and a Cβ atom that is chemically bonded to the Cα atom are added to the Cα atomic coordinates of step 50(1) and the objective function (Eₘₐᵢₙ, of formula (8) mentioned below) is minimized by the simulated annealing method. First, the Cα atoms are superposed three-dimensionally and the residues having a distance between Cα atoms of 2.5 Å or less are selected. The coordinates of main-chain atoms except the Cα are obtained from the coordinates of the reference proteins having the least distance between Cα atoms that should be superposed and are defined as the model structure.

In the case where there are no corresponding residues in the reference proteins, the main-chain atomic coordinates are constructed from a corresponding protein fragment composed of 4 residues in a database, which has been constructed beforehand (refer to, for example, K. Ogata and H. Umeyama, Journal of Molecular Graphics and Modeling 18, 258-272, 2000). In this procedure, the main-chain atom of the residue i is selected from the residues having the smallest rmsd value among Cα atoms from the (i−1)-th to (i+2)-th. At this time, the range of the superposition of the Cα atomic coordinates is from the i-th to (i+3)-th for the residues at the N terminal, and similarly from the (i−3)-th to i-th and from the (i−2)-th to (i+1)-th for the residues at the C terminal and one residue before the C terminal, respectively.

A main-chain atomic coordinates (also including a Cβ atom of a side-chain) is optimized by the simulated annealing method based on the objective function of the main-chain atom. The objective function is as the formula (8) below. One of the important differences between the conventional method and the present invention is that the chemical bond potential E_{bond}, the chemical bond angle potential E_{ang}, the chemical bond torsional angle potential Eₜₒᵣ and the interatomic interaction potential E_{non-bond} in the formula (8) are corrected as follows by k_{N} value (equal to the serial number of the C terminal residue of the N-th protein chain) determined by referring to the delimiter U in the alignment created in Step 40.
Eₘₐᵢₙ=E_{bond}+E_{ang}+Eₜₒᵣ+E_{non-bond}+Eₛₛ+Eₚₒₛ+E_{chi}+E_{hydr} (8)

E_{bond} is defined as the formula (9) below:

$${E}_{bond} = {K}_{b} {\sum }_{i} {( {b}_{i} - {b}_{i}^{0} )}^{2} \qquad ( 9 )$$

provided that, the case of i=k_{N(N=1,N-1)} and bᵢ=D_{Ci,Ni+1} is not added. (i+1 is the subscript of the subscript.)

Here, bᵢ⁰ is a standard bond length and varies depending on the three types of chemical bonds, N-Cα, Cα-C and C—N, however it is simplistically described here. K_{b} is a constant and set to, for example, 225. Regarding the calculation of E_{bond}, as shown in FIG. 5, because a chemical bond does not exist between the C terminal residue k_{N} of the N-th protein chain and the N terminal residue k_{N}+1 of the (N+1)-th protein chain, according to the conditions that bᵢ−bᵢ⁰=0 if i=k_{N(N=1,M-1)} and bᵢ=D_{Ci,Ni+1}, disconnection of interaction is performed as in the case of the calculation of Eₗₑₙ for Cα by excluding from the calculation of E_{bond}. In the case where the total number of protein chains is M, this procedure is performed from N=1 to M−1 and the residue numbers from k₁ to k_{M-1} can be specified according to the positions of the delimiter U from U₁ to U_{M-1} in the alignment created in Step 40.

E_{ang} is a function of chemical bond angle and as the formula (10) below:

$${E}_{ang} = {K}_{a} {\sum }_{i} {( {θ}_{i} - {θ}_{i}^{0} )}^{2} \qquad ( 10 )$$

provided that if i=k_{N(N=1,M-1)} and θᵢ=θ_{Cαi,Ci,Ni+1}, the case is not added, if i=k_{N(N=1,M-1)} and θᵢ=θ_{Di,Ci,Ni+1}, the case is not added, if i=k_{N(N=1,M-1)} and θᵢ=θ_{Ci,Ni+1,Cαi+1}, the case is not added. (explanation of three atoms that determine an angle)

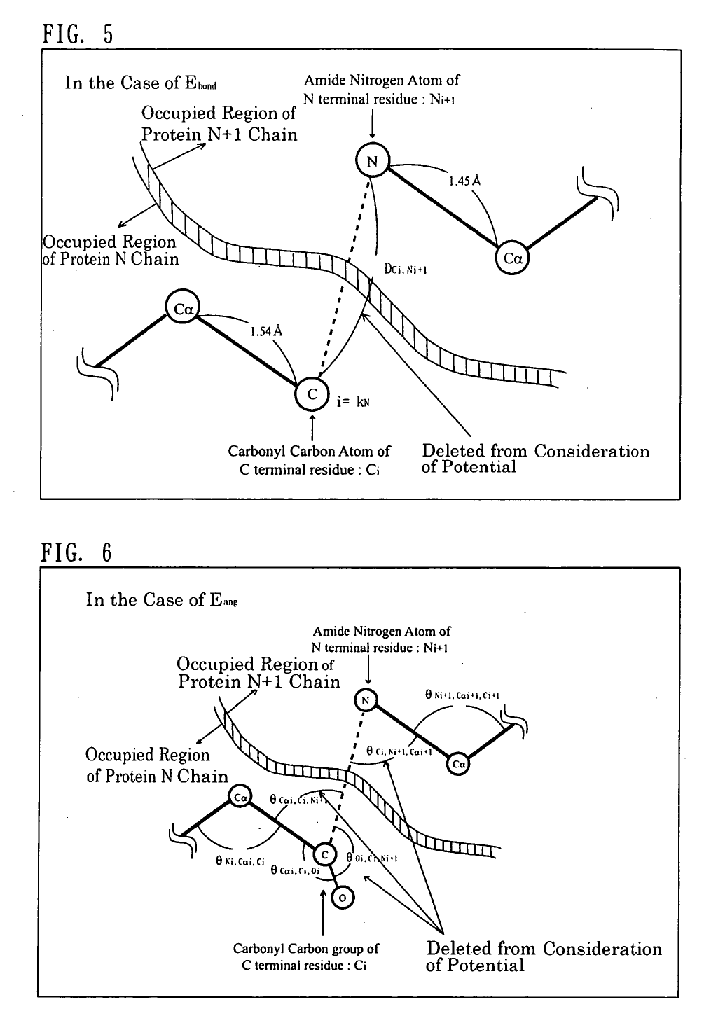

Here, θᵢ⁰ is a standard bond angle and varies depending on the types of each bond angle, however it is simplistically described here. Kₐ is a constant and set to, for example, 45. Regarding the calculation of E_{ang}, as shown in FIG. 6, because a chemical bond does not exist between the C terminal residue k_{N} of the N-th protein chain and the N terminal residue k_{N}+1 of the (N+1)-th protein chain, disconnection of interaction is performed by excluding from the calculation of E_{ang}. In the case where the total number of protein chains is M, this procedure is performed from N=1 to M−1 and the residue numbers from k₁ to k_{M-1} can be specified according to the positions of the delimiter U from U₁ to U_{M-1} in the alignment created in Step 40.

Eₜₒᵣ is the chemical bond torsional angle potential of a main-chain and is as the formula (11) below:

$${E}_{tor} = {K}_{t} {\sum }_{i} \sqrt{{( {ϕ}_{i} - {ϕ}_{i}^{0} )}^{2} + {( {ψ}_{i} - {ψ}_{i}^{0} )}^{2}} + {K}_{ω} {\sum }_{i} {( {ω}_{i} - {ω}_{i}^{0} )}^{2} \qquad ( 11 )$$

provided that if i=k_{N(N=1,M-1)}, φᵢ−φᵢ⁰=ωᵢ−ωᵢ⁰=φᵢ₊₁−φᵢ₊₁⁰=0.

Here, φᵢ⁰ and φᵢ⁰⁰ is set so that the torsional angle of the main-chain will satisfy Ramachandran plot. In other words, (φᵢ⁰, φᵢ⁰) that is closest to the coordinate (φᵢ, φᵢ) and satisfies Ramachandran plot is selected. In addition, ωᵢ⁰ is set to 0 and, is set to π(rad) only in the case of the cis-Pro residue. Kₜ and K_{ω} are constants and set to, for example, 10 and 50, respectively. Regarding the calculation of Eₜₒᵣ, as in the case of the calculation of E_{ang}, because a chemical bond does not exist between the C terminal residue k_{N} of the N-th protein chain and the N terminal residue k_{N}+1 of the (N+1)-th protein chain, disconnection of interaction is performed by excluding from the calculation of Eₜₒᵣ. FIG. 7 shows ωᵢ, and φᵢ and φᵢ₊₁ are handled in the same way. In the case where the total number of protein chains is M, this procedure is performed from N=1 to M−1 and the residue numbers from k₁ to k_{M-1} can be specified according to the positions of the delimiter U from U₁ to U_{M-1} in the alignment created in Step 40.

E_{non-bond} is the interatomic interaction potential and as the formula (12) below:

$${E}_{non - bond} = {K}_{non} {\sum }_{i , j} {ɛ}_{i , j}^{*} \{ {( \frac{{r}_{i , j}^{*}}{{r}_{i , j}} )}^{12} - 2 {( \frac{{r}_{i , j}^{*}}{{r}_{i , j}} )}^{6} \} \qquad ( 12 )$$

provided that if rᵢⱼ≦8.0 for rᵢⱼε{r_{Ni,Ni+1}; r_{Cαi,Ni+1}; r_{Cαi,Cαi+1}; r_{Ci,Ni+1}; r_{Ci,Cαi+1}; r_{Ci,Cβi+1}; r_{Ci,Ci+1}; r_{Oi,Ni+1}; r_{Oi,Cαi+1}} when i=k_{N(N=1,M-1)}, the case is included in the calculation of E_{non-bond}.

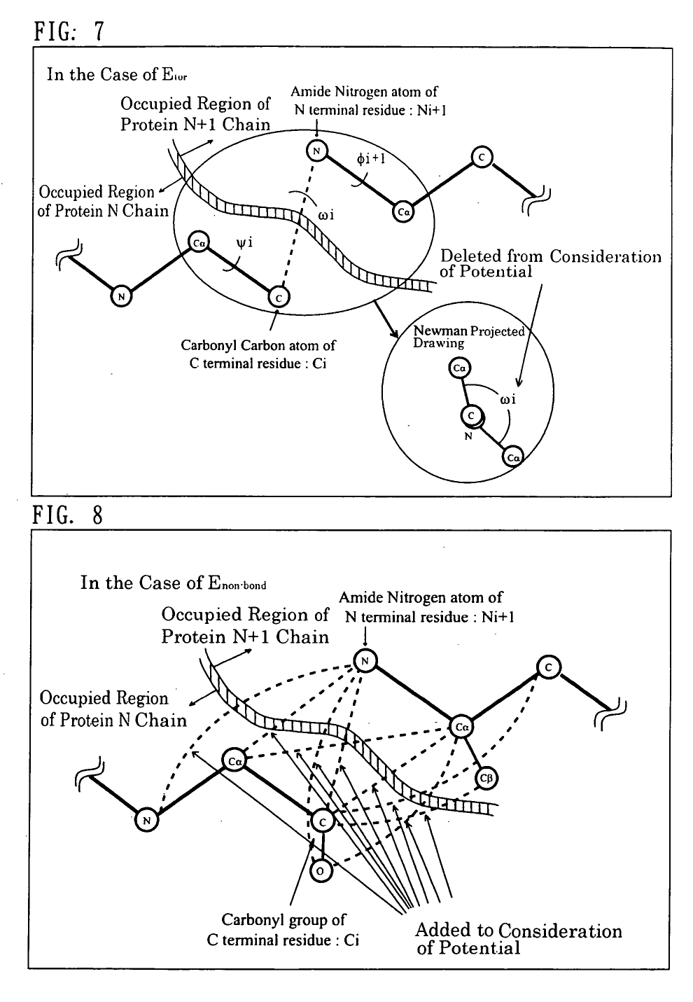

Here, εᵢⱼ* and rᵢⱼ* are constants that vary depending on the types of atoms (refer to Koji Ogata, doctoral dissertation, Tokyo University of Science, 1999). Kₙₒₙ is a constant and is set to, for example, 0.25, and it is normally included in the calculation of E_{non-bond}, in the case where the pairwise atoms i,j, whose rᵢⱼ is, for example, 8 Å or less, are separated by three or more bonds. Regarding the calculation of E_{non-bond}, as shown in FIG. 8, as in the case of the calculation of E_{vdw} for the Cα, because a chemical bond does not exist between the C terminal residue k_{N} of the N-th protein chain and the N terminal residue k_{N}+1 of the (N+1)-th protein chain, if rᵢⱼ belonging to {r_{Ni,Ni+1}; r_{Cαi,Ni+1}; r_{Cαi,Cαi+1}; r_{Ci,Ni+1}; r_{Ci,Cαi+1}; r_{Ci,Cβi+1}; r_{Ci,Ci+1}; r_{Oi,Ni+1}; r_{Oi,Cαi+1}} is 8 Å or less when i=k_{N(N=1,M-1)}, the case must be newly included in the calculation of E_{non-bond}. In the case where the total number of protein chains is M, this procedure is performed from N=1 to M−1 and the residue numbers from k₁ to k_{M-1} can be specified according to the positions of the delimiter U from U₁ to U_{M-1} in the alignment created in Step 40.

Eₛₛ is a function of a disulfide bond formed by the Cys residue and is as the formula (13) below.

$${E}_{SS} = {\sum }_{i} \{ {{K}_{C α}^{SS} ( {D}_{i}^{C α} - 5.4 )}^{2} + {{K}_{C β}^{SS} ( {D}_{i}^{C β} - 3.8 )}^{2} \} \qquad ( 13 )$$

Here, Dᵢ^{Cα} and Dᵢ^{Cβ} are the distances between the pairwise Cα atoms and the pairwise Cβ atoms of the Cys resides forming the disulfide bond within a protein chain or between protein chains respectively. Because the serial numbers throughout all the protein chains are used for the residue number i, in the present invention, Eₛₛ between protein chains can be handled with the conventional potential function. K^{ss}_{Cα} and K^{ss}_{Cβ} are constants and, for example, 7.5.

Eₚₒₛ is a function related to the positions of the main-chain atoms and is as the formula (14) below. The explanation of the formula is the same as that of the formula (6) mentioned above.

$${E}_{pos} = {K}_{pos} {\sum }_{i} \frac{1}{{M}_{i}} {‖ {x}_{i} - 〈 {w}_{i} {x}_{i} 〉 ‖}^{2} \qquad ( 14 )$$

Here, <wᵢxᵢ> is given as the formula (15) below. The explanation of the formula is the same as that of the formula (7) mentioned above.

$$〈 {w}_{i} {x}_{i} 〉 = \frac{1}{W} {\sum }_{j} {w}_{i}^{j} {x}_{i}^{j} \qquad ( 15 )$$

<wᵢxᵢ> in the above-mentioned formula (12) is obtained by superposing structures of the target protein and the reference protein. Kₚₒₛ is a constant and, for example, 0.3.

E_{chi} relates to chirality of Cα and is as the formula (16) below. Here, the chirality of Cα relates to an optical isomer of an amino acid residue (L-form or D-form), and the potential of the formula (16) is used so as to generally become a Cα atom of an L-form.

$${E}_{chi} = {K}_{chi} {\sum }_{i} {( {τ}_{i} - \frac{2}{3} π )}^{2} \qquad ( 16 )$$

Here, τᵢ is a torsional angle defined by N-Cα-Cβ-C of the i-th residue and K_{chi} is set to, for example, 50.

E_{hydr} relates to a hydrogen bond of the main-chain conserved in proteins with homologous sequences and is defined as the formula (17) below.

$${E}_{hydr} = {K}_{hydr} {\sum }_{i , j} {( {D}_{i , j}^{N - O} - 2.9 )}^{2} \qquad ( 17 )$$

A hydrogen bond is set when the distance of an N atom and an O atom is 2.9±0.5 Å. When it is judged whether a hydrogen bond exists in the plural reference proteins or not, it is judged to exist a hydrogen bond if at least 75% (at least 3 out of 4) of the reference proteins are confirmed to have a hydrogen bond. K_{hydr} is a constant and, for example, 0.6.

Next, the optimization of main-chain atoms including Cβ is performed by the simulated annealing method. In this procedure, the perturbation of the main-chain and Cβ atoms is set within 1.0 Å for the initial position. This procedure is generally repeated 200 times for the main-chain and Cβ atoms. The parameter corresponding to the temperature is generally started at 50 or 25, and decreased by a factor of 0.5 at every step until 0.01 is reached and then using a constant value for the parameter.

In order to take samples of a configuration of a main-chain widely, preferably, the above-mentioned method is performed six times and the main-chain atomic coordinates having the least value of the objective function (Eₘₐᵢₙ) is regarded as an optimal solution. Then the above parameter corresponding to the temperature generally is started at 50 for the first and the second of six optimizations and is started at 25 for the third and the rest.

Step 70: Construction and Optimization of Side-Chain Atomic Coordinates

The construction of a side-chain is roughly divided into two steps. It can be divided into “Construction of side-chain in structural conserved region” (Step 70(1)) and “Construction of all side-chains” (Step 70(2)) (refer to K. Ogata and H. Umeyama, Journal of Molecular Graphics and Modeling 18, 258-272, 2000).

Step 70(1): Construction of Side-Chain in Structural Conserved Region

For the calculated main-chain atom, in the case of structural conserved region (SCR), a torsional angle of a side chain is obtained from a protein with a homologous sequence using the method in the previous studies. The details of this method is described in “The role of played by environmental residues in side-chain torsional angles within homologous families of proteins: A new method of side-chain modeling.” Ogata K and Umeyama H, Prot. Struct. Funct. Genet. 1998, 31, 255-369. The whole content is included for reference in this description as a part.

In this method, the ratio of side-chains conserved in the proteins with homologous sequences is calculated, and the modeling of side-chains is performed based on this information. The side-chain atomic coordinates in a conserved region of a side-chain is located to the fixed main-chain atom. For example, if the χ¹ angle of an arginine residue in a protein with a homologous sequence is conserved, the Cγ atomic coordinates can be located, and if χ¹ and χ² angles of a Phe residue are conserved, all the side-chain atoms can be located. The optimization step of the simulated annealing using the formula (8) is performed only for the main-chain and the Cβ atoms, and the perturbation of the atoms was set within 1.0 Å. This annealing step of the main-chain and the Cβ atoms is repeated 200 times. The parameter corresponding to the temperature is started at 25 and decreased by a factor of 0.5 at every step until 0.01 is reached. E_{non-bond} in the above-mentioned formula (8) is performed for the main-chain atom and the partially constructed side-chain atoms. At this time, the side-chain atomic coordinates are made to be conserved throughout the optimization step.

Mi in the above-mentioned formula (14), which is the information regarding structure, and a pair of N—O forming a hydrogen bond in the above-mentioned formula (17) are re-calculated for the distance in the optimization step. In particularly an N—O pair is used depending on the judgment of existence of a hydrogen bond. In order to obtain the configuration of the main-chain atom, the above-mentioned procedure is repeated 3 times and the main-chain atomic coordinates having the least value of the objective function is determined as the calculated structure.

Step: 70 (2): Construction of All Side-Chains

The construction of all the side-chains is performed in the group of the fixed main-chain and Cβ atoms. This is performed according to the method described in the above-mentioned literature of Ogata K and Umeyama H, Prot. Struct. Funct. Genet. 1998, 31, 255-369. By using the method, an accurate model can be given in a short time. First, a main-chain structure (including Cβ) is optimized at low temperature by the Monte Carlo method using the objective function Eₘₐᵢₙ of the above-mentioned formula (8). At this time, the temperature is set at 0.001 and regarding E_{non-bond} in the above-mentioned formula (8), the calculation between the main-chain atoms and all the side-chain atoms is performed. Then, the side-chain coordinates are re-located so as to maintain the torsional angle of the side-chains at the optimized state in the optimization step of N, Cα, C and Cβ atoms. The perturbation of atoms is set within 0.5 Å. Next, the side-chains are deleted and the above-mentioned construction of the side-chains is repeated. This procedure is repeated until the constructed structure does not have a collision of atoms of 2.4 Å and the torsional angle of N-Cα-Cβ-C is fallen within the range of −120±15°.

Step 80: Evaluation of Appropriateness of Model Structure

The evaluation of appropriateness of the tertiary structure of a completed model is performed by superposing the main-chain atomic coordinates of the model and the reference protein for regions excluding a loop insertion or a deletion region, and the model was judged appropriate if rmsd is 1 Å or less.

Step 90: Construction of Final Structure: Tertiary Structure Prediction

As mentioned above, based on the alignment obtained in Step 40, a tertiary structure is constructed using a modeling software such as newFAMS that was newly developed by the present inventors this time in Steps 50-80, and then a model is completed. Together, the method shown in the above-mentioned Steps 40-80 is referred to as “newFAMS”. On the other hand, the conventional modeling software (refer to K. Ogata and H. Umeyama, Journal of Molecular Graphics and Modeling 18, 258-272, 2000), which is the basis for the present invention, is simply referred to as “FAMS”.

The single chain modeling formulae has been corrected as described in this description of the above-mentioned formulae (2), (3), (4), (9), (10) and (12) to realize the temporary single chain modeling of the plural chains modeling. As another method, the temporary single chain can be realized by handling the corresponding coefficients of these formulae, Kl of the above-mentioned formula (2), Ka of the formula (3), Kvdw of the formula (4), Kb of the formula (9), Ka of the formula (10) and Kt of the formula (11) as values dependent on the residue number, and adjusting them respectively. At the step in which the present invention is specifically implemented, this temporary single chain modeling can be derived easily from the constitutions of the right side of the above-mentioned formulae (2), (3), (4), (9), (10) and (12). In addition, this temporary single chain modeling gives exactly the same results as the below-mentioned Examples 1 to 4.

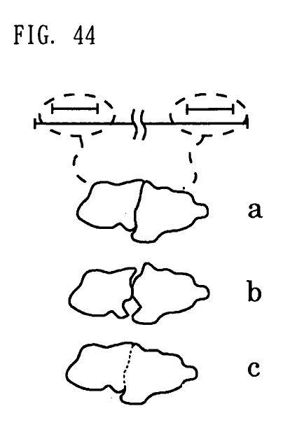

According to the present invention, a tertiary structure in which domains (plural chains) separated in the sequence or separated widely are contacted in the space each other can be selected as a reference protein (refer to FIG. 44). A tertiary structure of a complex in the state where domains (plural chains) separated in the sequence or separated widely, are contacted in the space each other is determined experimentally (refer to FIG. 44a). By performing the modeling by the present invention using the determined tertiary structure as a reference protein, the tertiary structure of the target protein can be constructed accurately (the method of the present invention; refer to FIG. 44c). On the other hand, if the modeling is similarly performed by the conventional method, a tertiary structure, in which the contact surface of both domains is inaccurate, is constructed as shown in FIG. 44b. In such construction of tertiary structure, it is understood that more accurate tertiary structure can be constructed according to the present invention compared to the conventional method.

As mentioned above, the whole content of the literature about the conventional method cited in this description is included for reference in this description as a part. Similarly, the whole content regarding the invention of application documents such as the specification included in the Japanese application applied on Jan. 9, 2002: JP-A2002-2859, which is the basis for the present application, is included for reference in this description as a part.

### Preferred Embodiments

Hereinafter, the present invention will be described in detail with reference to the following examples. These are only for the purpose of illustrating the present invention, therefore the present invention is not limited these examples.

### EXAMPLE 1

### Example of Modeling in Metabotropic Glutamate Receptor Family

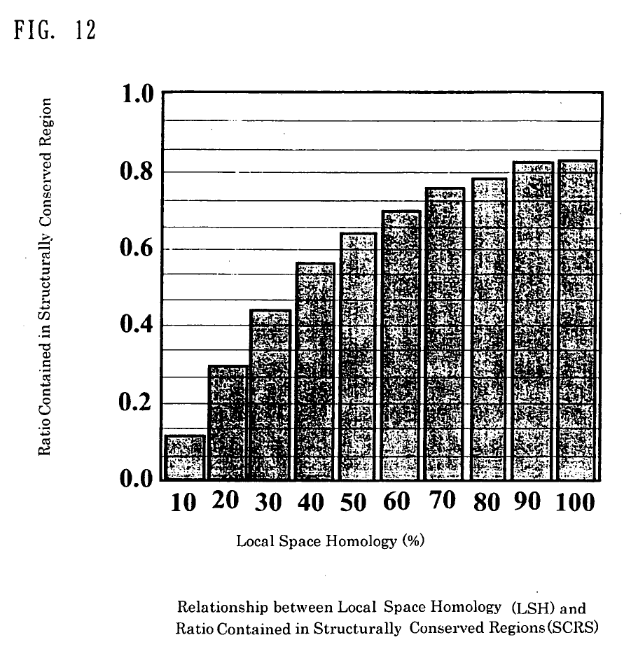

The primary amino acid sequence of metabotropic glutamate receptor 1 protein derived from rat (reference protein) was obtained from Swiss-Prot (entry name MGR1_RAT, accession number P23385). As shown in FIG. 13 (refer to SEQ ID NO:1 in the sequence listing), it has been found by an X-ray structure analysis by Morikawa et al. that the whole is 1199 residues, among the former part of 478 residues indicated by underlining, the 9 residues located from the 448th to 456th become the contact region each other, a dimmer (homodimer) is formed by assembling two monomers, and the glutamate receptor domain is formed (refer to Kunishima, N., Shimada, Y., Tsuji, Y., Sato, T., Yamamoto, M., Kumasaka, T., Nakanishi, S., Jingami, H., Morikawa, K.: Structural Basis of Glutamate Recognition by a Dimeric Metabotropic Glutamate Receptor, Nature 407, 971, 2000). Three types of protein tertiary structures (1EWK, 1EWT, 1EWV) are registered in PDB. 1EWK is a structure including glutamate as a ligand and 1EWT is a structure not including glutamate as a ligand. From the result of an X-ray structural analysis, it has been known that 1EWK is a closed form in which monomers are relatively close to each other, on the other hand, 1EWT is an open form in which monomers are relatively separated each other. Both are different in the relative configuration of the monomers in this way. Therefore the states of the bound surfaces between domains are significantly different.

That is, the glutamate receptor 1 protein has the open form in the state where the ligand is not bound (1EWT), but has the closed form in the state where the ligand is bound (1EWK), which is presumed to be more stable. Therefore, in this protein, it is very important to accurately perform modeling of the state of the bound surface including relative configuration of monomers for the elucidation of the function. Consequently, as an example, using 1EWT as a reference protein for the search of a homologous sequence, the tertiary structure model of a protein dimer, which does not include a ligand (reference protein), was constructed. In addition, it was examined if relative merits and demerits of the tertiary structure (difference in an energetic stability) occurred in the state of the bound surface between dimers by comparing the case where the modeling of each monomer, which constitutes a dimer, was performed by the conventional method and both were combined to make a dimer model with the case where the modeling of a dimer itself was performed by the present invention.

First, using the 1199 residues of MGR1_RAT amino acid sequence as a query, using PIR as of November 2001 as a motif profile, a PSI-BLAST search was performed on 774804 sequences registered in protein amino acid sequence databases such as PIR, Swiss Prot, TREMBL, TREMBL_NEW, GenPept (all as of November 2001). As a result of the search performed under the condition where E-value is 0.001 or less, 14509 homologous sequences and alignments were obtained. Among them, there are 70 whose E-value is 0 (the homology is between 23 and 100%, and because E-value is very small it is described as 0 as the computer output). These can be considered almost identical regarding function. Among them, a modeling of the protein dimer of a receptor, derived also from rat, whose Swiss Prot entry name is “MGR5_RAT” and accession number is “P31424” (target protein), was performed (refer to Abe T., Sugihara H., Nawa H., Shigemoto R., Mizuno N., Nakanishi S., Molecular characterization of a novel metabotropic glutamate receptor mGluR5 coupled to inositol phosphate/Ca2+ signal transduction, J. Biol. Chem. 267, 13361-13368, 1992). MGR5_RAT is a signal transduction protein related to inositol phosphate and calcium ion, and is a metabotropic glutamate receptor protein subtype 5 derived from rat, and its amino acid residue number is 1203. The homology between the reference sequence MGR1_RAT and the target sequence MGR5_RAT is 62.2% and the result of their alignment is shown in FIG. 14 (refer to SEQ ID Nos:1 and 2 in the sequence listing).

(Result of Alignment Between MGR1_RAT and MGR5_RAT)

| | | | | | | |
|---|---|---|---|---|---|---|
| >MGR5_RAT | 1203 | homology | match | mismatch | in- | de- |
| | | | | | sertion | letion |
| >MGR1_RAT | 1189 | 62.2% | 740 | 396 | 53 | 67 |

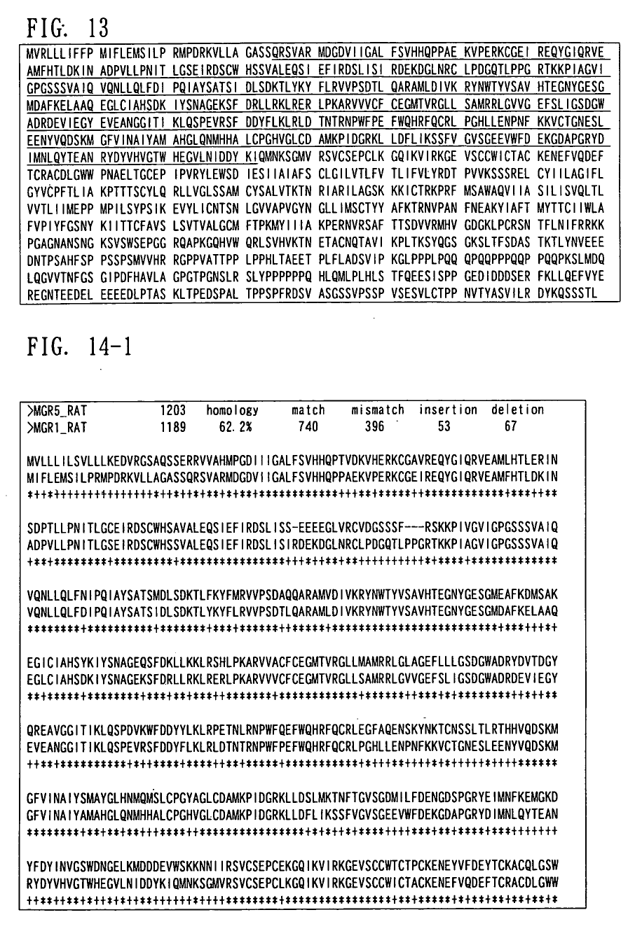

FIG. 15 only shows regions in which coordinates of 1EWT in PDB exist in the alignment of FIG. 14 (refer to SEQ ID Nos:3 and 4 in the sequence listing). A modeling was performed using this alignment.

(Result of Alignment Between MGR1_RAT and MGR5_RAT; Only Regions in which Coordinates Exist in 1EWT)

| | | | | | | |
|---|---|---|---|---|---|---|
| >MGR5_RAT | 474 | homology | match | mismatch | in- | de- |
| | | | | | sertion | letion |
| >1EWT_A | 456 | 73.9% | 337 | 118 | 1 | 19 |

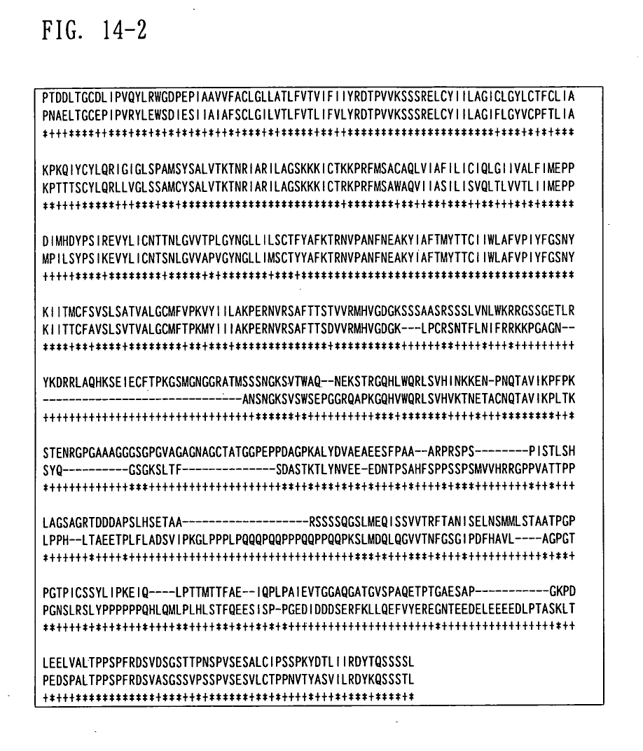

The alignment in FIG. 15 is for a monomer and the input file format in the conventional FAMS (refer to K. Ogata and H. Umeyama, Journal of Molecular Graphics and Modeling 18, 258-272, 2000) is as shown in FIG. 16 (refer to SEQ ID Nos:3 and 4 in the sequence listing). This modeling was performed by using the conventional FAMS. The modeling result is shown in FIG. 17.

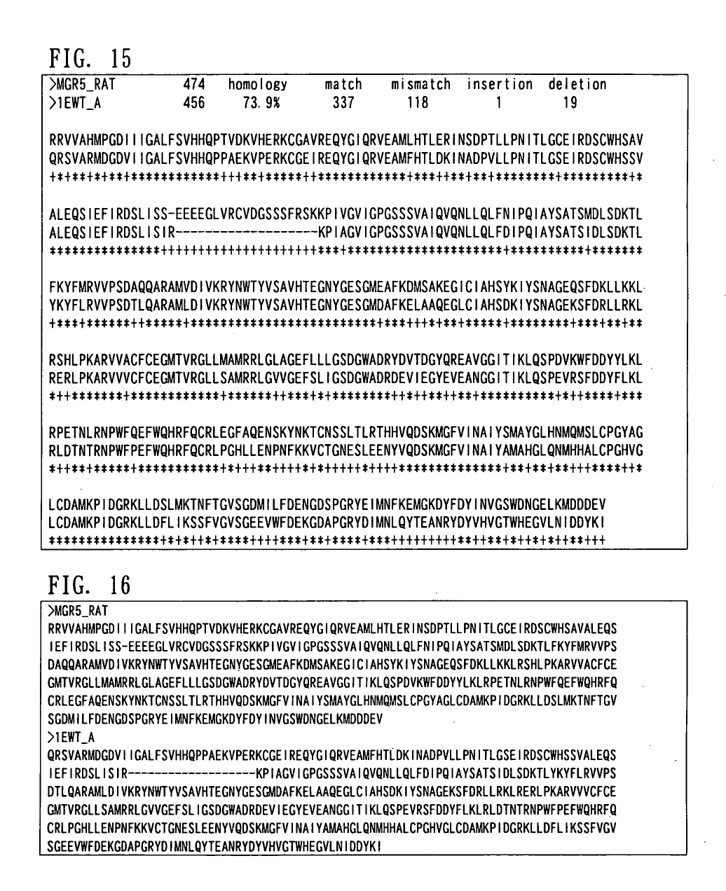

Further, in the case of handling the alignment as a homodimer, the alignment in FIG. 16 is connected with the character “U”, which is as shown in FIG. 18. This input file format, in which “U” is used, is the one which has been developed by the present inventors. The modeling result is shown in FIG. 19.

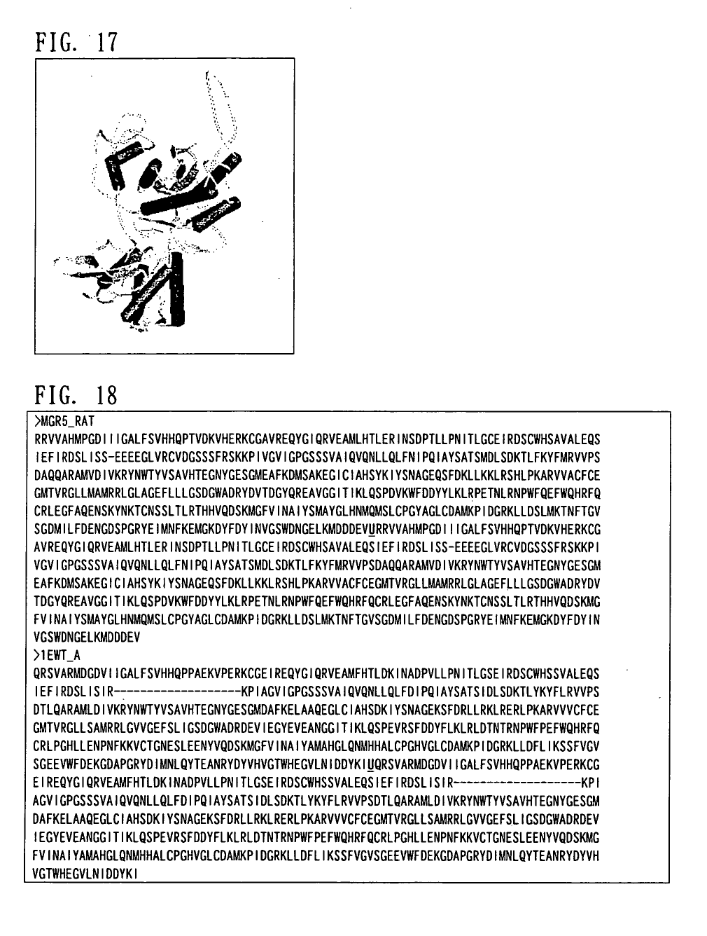

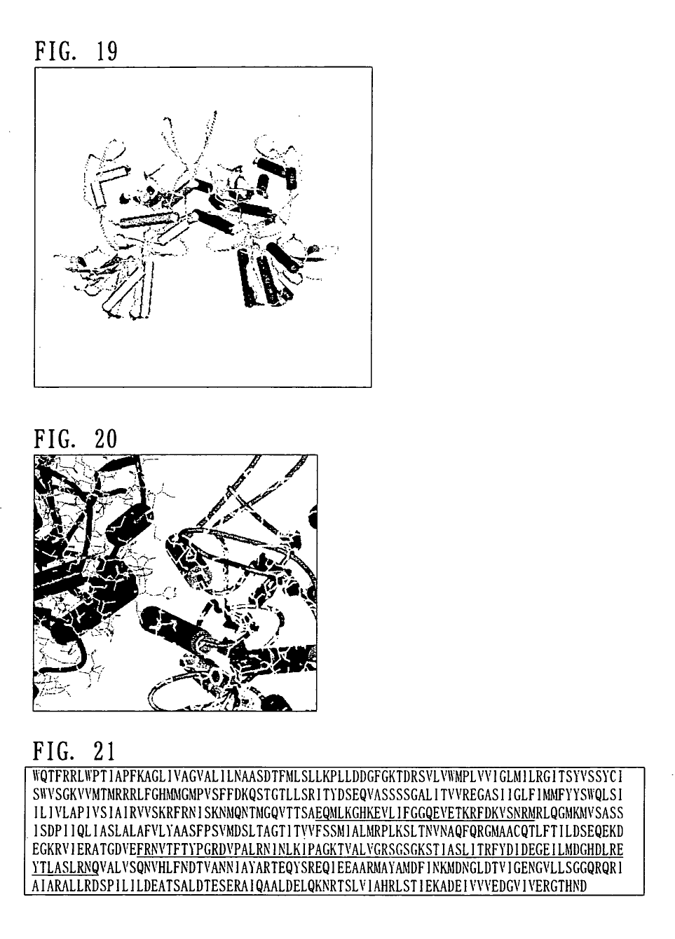

Further, the enlarged drawing of the bound surface of a homodimer model by newFAMS of FIG. 19 is shown in FIG. 20. In this drawing, the modeling of the recognition region of protein-protein interaction is precisely performed without a collision within 2.4 Å in a main-chain or a side-chain. On the other hand, when a tertiary structure of each of MGR5_RAT was constructed independently with the conventional FAMS using the three-dimensional coordinates of each monomer of 1EWT homodimer, interatomic contact within 2.4 Å occurred at 8 positions on the bound surface. This structure is quite energetically unstable because lots of collisions occur at the bound surface. Meanwhile, the model structure by newFAMS is energetically stable because no collision occurs at the bound surface. This indicates the superiority (novelty) of newFAMS for performing a modeling of plural chain, which the present inventors have developed.

### EXAMPLE 2

### Modeling Example of Transporter

The tertiary structure of a homologue of ABC transporter, which is believed to be one of the causes of multidrug resistance, has been analyzed by an X-ray crystallography at the resolution of 4.5 Å and registered, although only for Cα coordinates, in PDB as 1JSQ (reference protein). This tertiary structure suggestes that ABC transporter form a homodimer on plasma membrane and have a function to release a phospholipid from a cell by the flip-flop movement of relative position between monomers (refer to Geoffrey Vhang and Chistopher B. Roth, SCIENCE, Vol 293, pp. 1793).

1JSQ is composed of 8 chains (A chain, B chain, C chain, D chain, E chain, F chain, G chain and H chain) and is registered as 4 combinations of homodimers of A-B chains, C-D chains, E-F chains and G-H chains. In this example, first, a main-chain and a side-chain were constructed for the respective 8 chains only from Cα coordinates by the automatic modeling of the conventional FAMS. From the observation of the eight coordinates constructed up to side-chains, the chemical bond torsional angles φ and φ for the main-chain of B chain was the structure that least went into the energetically unstable region on a Ramachandran plot. Next, by the chimera modeling method (refer to T. Yoneda, H. Komooka, H. Umeyama, J. Prot. Chem., 16, 597-605, 1997), using B chain as a basic structure, a monomer structure modeling was performed by partially supplementing with the other chains. Then, the coordinates of B chain monomer were rotated, transferred and superposed on those of A chain and the coordinates after the transfer were defined as a new A chain. The A-B chain pair thus obtained was used in the below-mentioned modeling as a template structure MSBA (modeled reference protein). The amino acid sequence of 1JSQ monomer contains 555 residues (two underlined regions are coordinate deletion and it contains 450 residues if these are excluded) as shown in FIG. 21 (refer to SEQ ID NO:5 in the sequence listing).

Using this sequence of 555 residues as a target sequence (query) to be referred to, the motif profile of the query was created using PIR database as of November 2001, and IMPALA search (although it is similar to PSI-BLAST, it is a method using the alignment by the Smith and Waterman method: refer to A. A. Schaffe et al., BIOINFORMATICS, 15(12), 1000-1011, 1999) was performed for 774804 sequences in the protein amino acid sequence databases such as PIR, Swiss Prot, TREMBL, TREMBL_NEW and GenPept (all as of November 2001). As a result of the search under the condition where the E-value is 0.001 or less, alignments with 13705 homologous sequences were obtained.

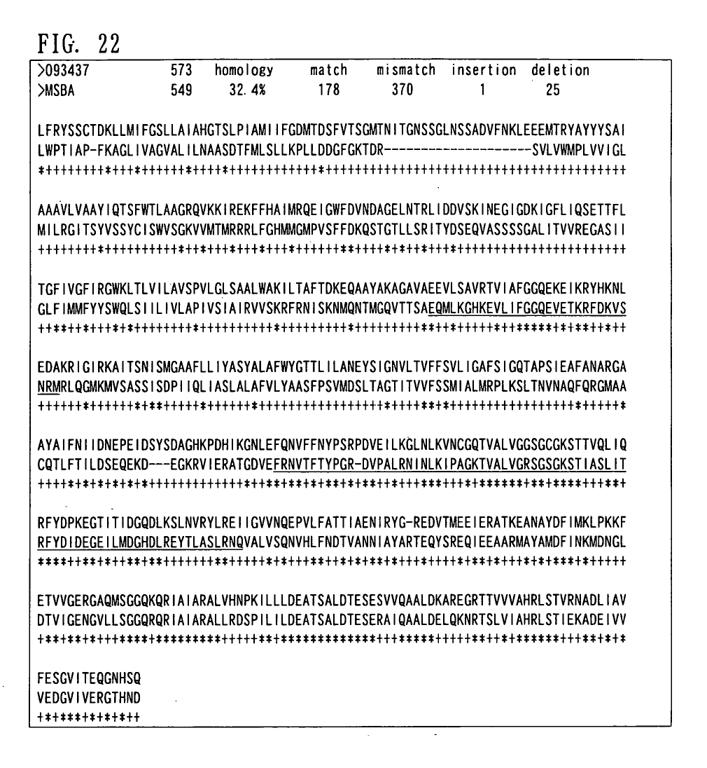

As an example, a modeling was performed on ID “O93437” in Swiss Prot database. O93437 (target protein) is described as ABC transporter protein in chicken in Swiss-Prot homepage (refer to Edelmann H. M. L., Duchek P., Rosenthal F. E., Foeger N., Glackin C., Kane S. E., Kuchler K., “Cmdrl, a chicken P-glycoprotein, confers multidrug resistance and interacts with Estradiol”, Biol. Chem. 380, 231-241, 1999). The number of amino acid residues is 1288 and it is a protein showing multidrug resistance and interaction with Estradiol, which is an estrogenic hormone. In order to perform a modeling of O93437, the result of the alignment with the above-mentioned MSBA is shown in FIG. 22 (refer to SEQ ID Nos: 6 and 7 in the sequence listing).

(Alignment Result of “O93437” and MSBA)

| | | | | | | |
|---|---|---|---|---|---|---|
| >O93437 | 573 | homology | match | mismatch | insertion | de- |
| | | | | | | letion |
| >MSBA | 549 | 32.4% | 178 | 370 | 1 | 25 |

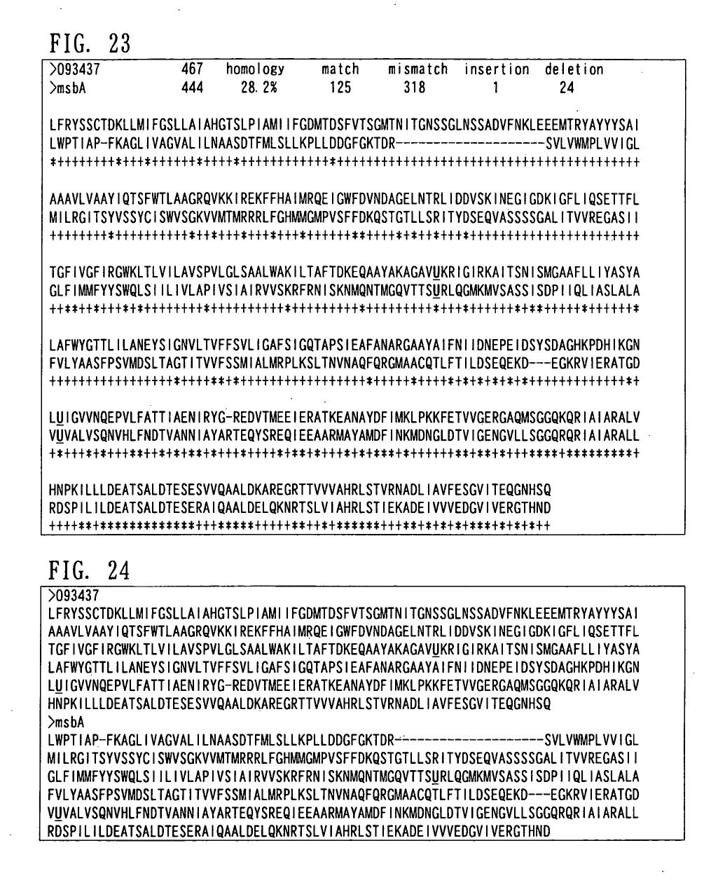

However, because MSBA, which is the reference protein, has two large coordinate deletions within it (the regions indicated by underlining in 1JSQ amino acid sequence in FIG. 21), a modeling cannot be performed with the alignment as it is in FIG. 22. Therefore, the character “U” was inserted in the deleted coordinate regions and the alignment was corrected as shown in FIG. 23. This is for describing a protein as if it were composed of three proteins and performing a modeling for plural proteins.

(New Alignment Result of “O93437” and MSBA)

| | | | | | | |
|---|---|---|---|---|---|---|
| >O93437 | 467 | homology | match | mismatch | insertion | deletion |
| >msbA | 444 | 28.2% | 125 | 318 | 1 | 24 |

This becomes as shown in FIG. 24 when it is changed to the input file format for newFAMS.

Although in the alignment in FIG. 24, the protein is handled as a temporary single chain and a protein model is constructed, this input file is insistently for a monomer. The modeling result using this alignment is shown in FIG. 25.

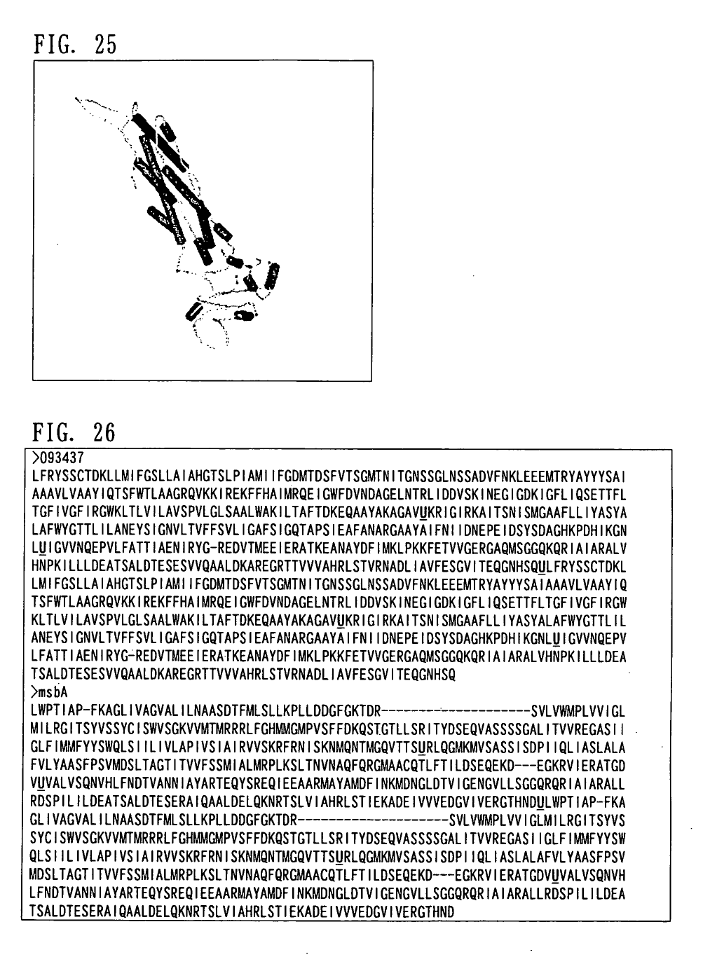

Next, in the case where a model is constructed as a homodimer, the above alignment is further connected with the character “U” and becomes as shown in FIG. 26 below.

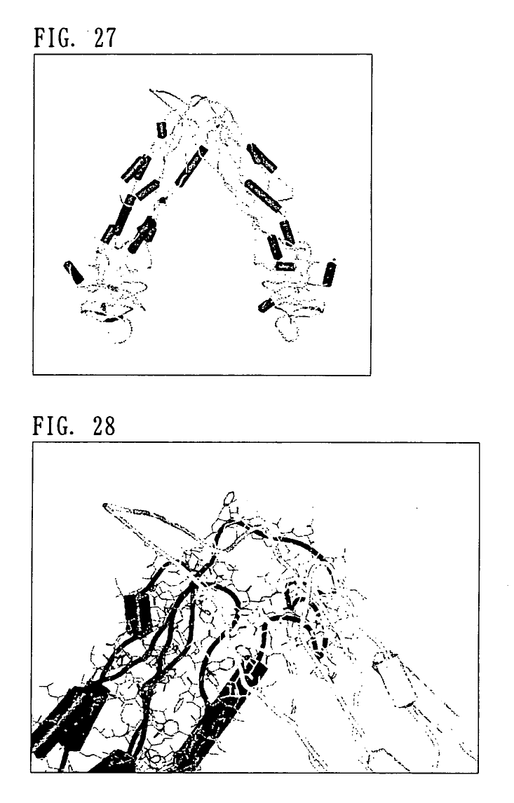

The modeling result by newFAMS using the input file format of FIG. 26 is shown in FIG. 27.

The graphic display of the contact region of the above-mentioned homodimer is as shown in FIG. 28.

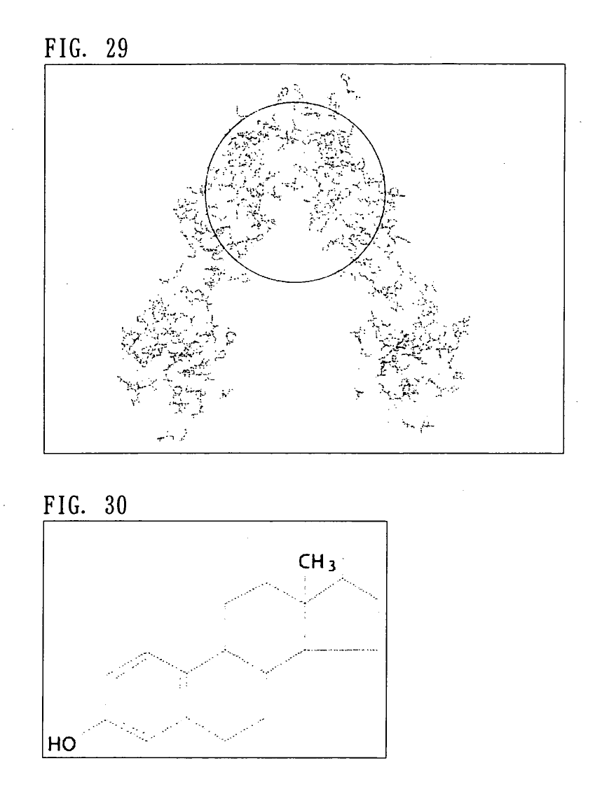

In this way, even in the case where a coordinate deletion exists, by handling it as temporary plural chains and replacing with the character “U”, the calculation of structure, in which a collision of a van der Waals atom is considered, becomes possible and a homodimer model that represents accurately the recognition region of interaction can be constructed. In the observation of the constructed homodimer model “O93437”, the region, which is considered as the transmembrane region of the homodimer from an X-ray analysis and where a substance to be transported is mentioned to possibly bind, is presumed to be the region enclosed with a circle in FIG. 29. In this region, a considerable number of hydrophobic amino acid residues exist and an estrogenic hormone estradiol, which is believed to be the function of “O93437”, is presumed to be able to bind easily. By the way, the estradiol, which interacts, is composed of a hydrophobic group throughout the molecule as shown in FIG. 30. On the other hand, when each model construction of “O93437” ABC transporter was independently performed using the three-dimensional coordinates of each monomer of the homodimer in which a model construction of a main-chain and a side-chain was performed based only on the Cα atomic coordinates described in the above-mentioned 1JSQ as a reference protein, and the recognition region of interaction was observed, because the interaction between the homodimers was not taken into consideration, interatomic contact within 2.4 Å occurred at 54 locations on the bound surface. As mentioned above, because the dimer contact region is located close to the region where estradiol of a ligand possibly binds, the model construction of the bound region of this homodimer is extremely important for describing the function accurately. This is also considered to indicate the superiority (novelty) or usefulness of the plural chain modeling method in the present invention.

### EXAMPLE 3

### Example of Modification of Peptide Binding to Lethal Factor Protein of Anthrax

The tertiary structure of lethal factor (LF, molecular weight of 90,000) that is the toxin of anthrax, which has made a sensation in these few years, was reported in the November 2001 issue of the British journal Nature (Pannifer et al., NATURE, vol 414, pp. 229-233) at the resolution of 3.90 Å by an x-ray crystallographic analysis. This protein is composed of four domains and is an essential enzyme for the pathogenicity of anthrax, and inhibits one or more signal transduction systems of human cells. Specifically, it has been reported to contact with the N terminal region of a protein family called mitogen-activated protein kinase kinase (MAPKK) and cleave the terminal region. LF is a protease with significantly high specificity. In PDB, LF itself is registered as 1J7N, and the complex of LF and the 16 residues in the N terminal region of a MAPKK family called MAPKK-2 is registered as 1JKY (reference protein). The 16 residues in the N terminal region of MAPKK-2 are cleaved by being caught in a long and deep groove formed with the three domains II, III and IV of LF. Because a drug targeted to this toxin is considered promising, it is possible to design a drug, which is not a peptide, by referring to the tertiary structure of the 16 residues in the N terminal region of MAPKK-2.

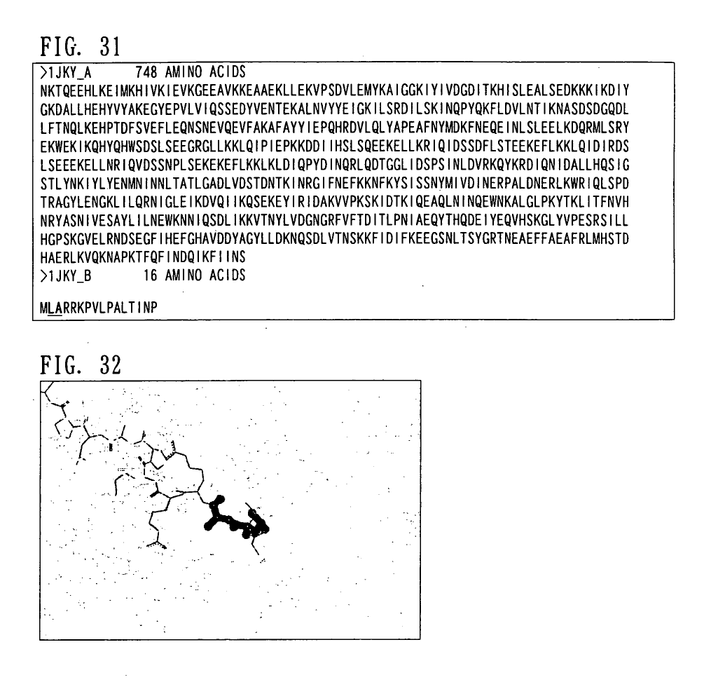

In this example, a model of a sequence having a hypothetical mutation in the 16 amino acid residues of MAPKK-2 (target protein) was constructed, and changes of the interaction mode between these models and LF was shown. Generally, drug design focuses on a hydrophobic binding region because drug absorption from the intestinal tract and the like need to be considered. PDB: 1JKY contains A chain (LF) and B chain (16 residues of MAPKK-2) and the amino acid sequences are shown in FIG. 31 (refer to SEQ ID Nos: 8 and 9 in the sequence listing).

In the 16 residues of MAPKK-2 of B chain, there is a spatial gap between the region of the second and the third residues “LA” and LF of A chain (refer to FIG. 32). Therefore, by replacing an amino acid residue in this region with an amino acid residue having a more bulky side-chain and designing such an inhibitor so as to further strengthen the contact by a hydrophobic interaction, the inhibitor binds to the LF in a competitive manner with the 16 residues in the N terminal of MAPKK-2, thereby it may be possible to inhibit the activity of LF as a protease.

A model in which, for example, “FF” is selected as an amino acid residue having the more bulky side-chain instead of “LA” was constructed.

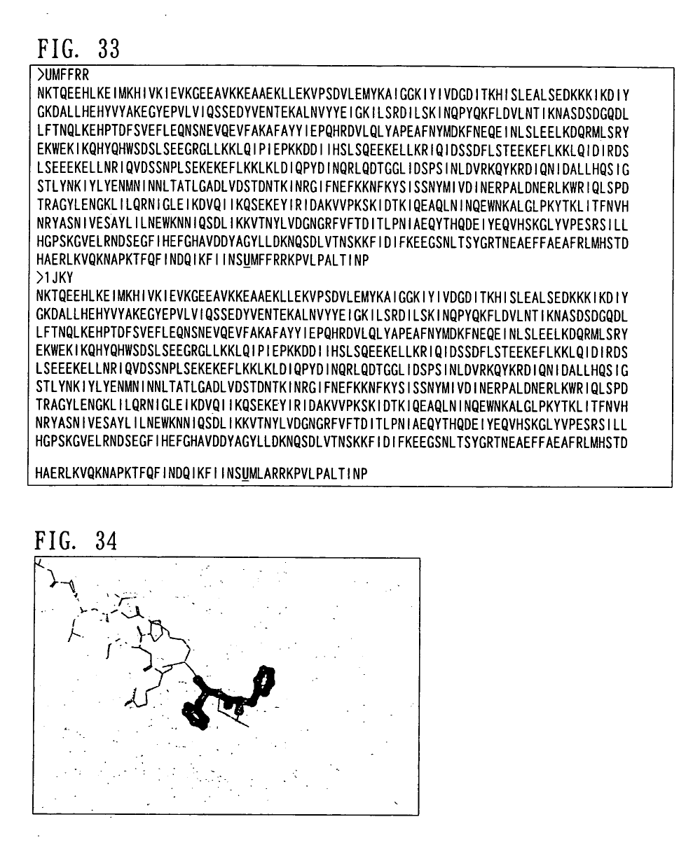

Using the complex (1JKY) of this A chain (LF) and B chain (MAPKK-2) as a template, with the use of the input file format of FIG. 33 (refer to SEQ ID NO:10 in the sequence listing), a model construction was performed by newFAMS. The modeling result is shown in FIG. 34.

As above, an example is shown for the case where a model construction was performed by replacing “LA” with “FF”. However, it is possible to analyze other mutants comprehensively using newFAMS and it becomes possible to assume a lead compound of a more effective inhibitor among them.

### EXAMPLE 4

### Verification Example of the Present Invention by Comparison of Tertiary Structure of Protein Composed of Plural Chains Actually Registered in PDB and Constructed Model (Blind Test)

The tertiary structure of the complex protein 2PTC of trypsin, which is a kind of proteinase, and its protein inhibitor, pancreatic trypsin inhibitor (PTI), has been analyzed at the resolution of 1.90 Å by an X-ray crystallographic analysis and registered (refer to Marquart, M., Walter, J., Deisenhofer, J., Bode, W., Huber, R.: The Geometry of the Reactive Site and of the Peptide Groups in Trypsin, Trypsinogen and its Complexes with Inhibitors, Acta Crystallogr., Sect. B 39, 480, 1983). 2PTC has been registered as a complex composed of E chain of trypsin and I chain of PTI. Trypsin, which is a serine protease, is secreted from pancreas as an inactive trypsinogen and becomes an active form trypsin by an enzyme, Enterokinase, contained in duodenal juice. Trypsin is a kind of end peptidase and a protein digesting enzyme which cleaves a peptide bond at the carboxyl group side of a basic amino acid such as Arg or Lys.

On the other hand, the tertiary structure of the complex protein 1BTH of thrombin E192Q enzyme, which is a kind of serine protease (glutamic acid 192 is replaced with glutamine) and PTI has been analyzed at the resolution of 2.3 Å by an X-ray crystallographic analysis and is registered (refer to van de Locht, A., Bode, W., Huber, R., Le Bonniec, B. F., Stone, S. R., Esmon, C. T., Stubbs, M. T., “The thrombin E192Q-BPTI complex reveals gross structural rearrangements: implications for the interaction with antithrombin and thrombomodulin” EMBO J. 16, 2977, 1997). This protein has been registered as the form of two sets of homodimers, thrombin E192Q composed of L chain and H chain and PTI composed of only P chain, and similarly, thrombin E192Q composed of J chain and K chain and PTI composed of only Q chain. Here, the former set of the protein complex is focused. Thrombin is an active form protein of prothrombin, which is one of the blood coagulation factors, and by forming this thrombin, fibrinogen becomes fibrin and blood coagulation is caused.

In this example, a modeling of H chain and P chain of 1BTH was performed from E chain and I chain of 2PTC using newFAMS, which is the software of the present invention, and a comparison was made with the X-ray structure of 1BTH actually registered in PDB (Protein Data Bank). However, 1BTH itself is not included in the database of Cα atoms and main-chain atoms used for the construction of the inserted loop of 1BTH model, and it was confirmed that a database including the structure of the right answer was not used in the verification of the model accuracy. Further, a comparison with the modeling of each 1BTH_H and 1BTH_E using the conventional FAMS is shown to verify the accuracy of newFAMS. What should be noted here is that, in H chain of 1BTH, the 192nd amino acid residue of thrombin is replaced from a glutamic acid to a glutamine. The 192nd amino acid residue of thrombin is glutamic acid, and generally the loop region of the enzyme sterically hinders to bind to PTI. However, if the 192nd amino acid residue is replaced with a glutamine (thrombin E192Q), when binding to PTI, the above-mentioned hindered loop region moves by induced-fit adaptation being affected by PTI, the both proteins bind to each other. This is the complex protein registered as the name of 1BTH.

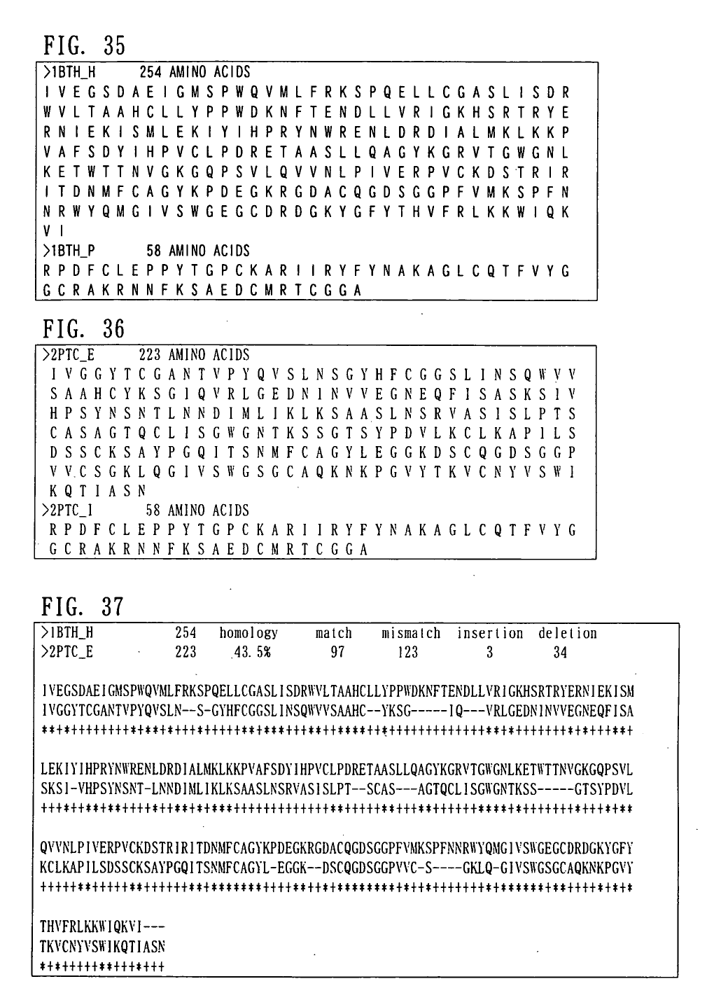

The amino acid sequences of H chain and P chain of 1BTH, which is the target protein, are as shown in FIG. 35 (refer to SEQ ID Nos:11 and 12 of the sequence listing). The numbers of amino acid residue are 254 for H chain and 58 for P chain in 1BTH.

Oh the other hand, the amino acid sequences of E chain and I chain of 2PTC, which is the reference protein, are as shown in FIG. 36 (refer to SEQ ID NOs:12 and 13 of the sequence listing). The numbers of amino acid residues are 223 for E chain and 58 for 1 chain in 2PTC.

The alignment of H chain of 1BTH and E chain of 2PTC is as shown in FIG. 37. The homology was 43.5%.

(Alignment Result of H Chain of 1BTH and E Chain of 2PTC)

| | | | | | | |
|---|---|---|---|---|---|---|
| >1BTH_H | 254 | homology | match | mismatch | insertion | de- |
| | | | | | | letion |
| >2PTC_E | 223 | 43.5% | 97 | 123 | 3 | 34 |

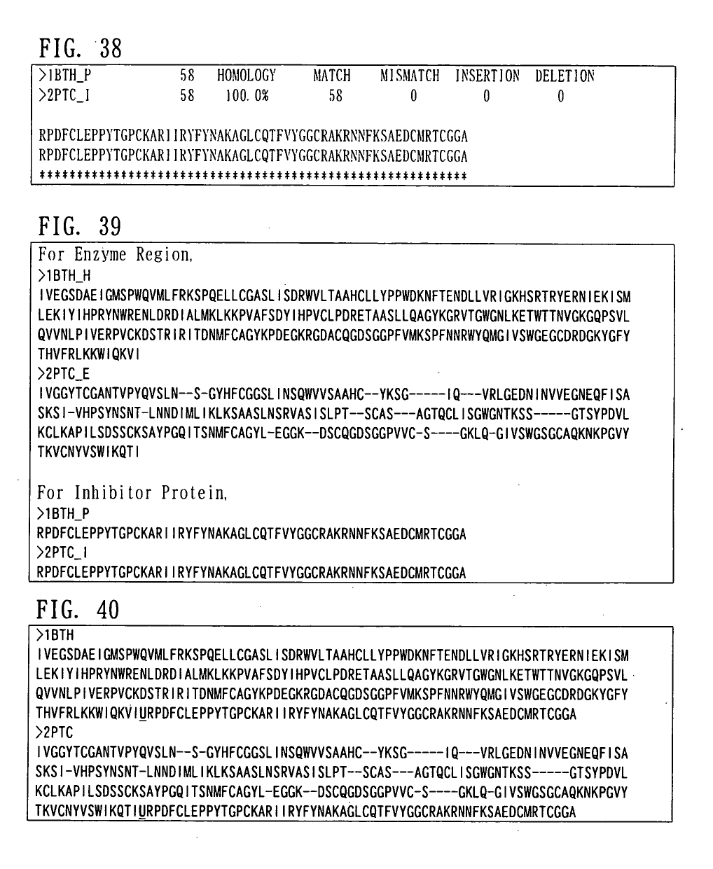

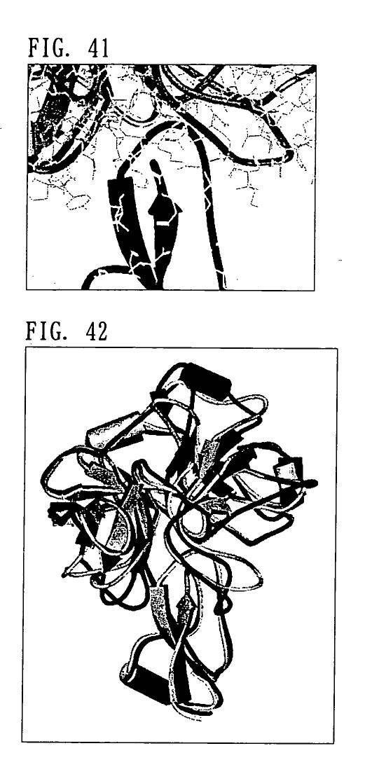

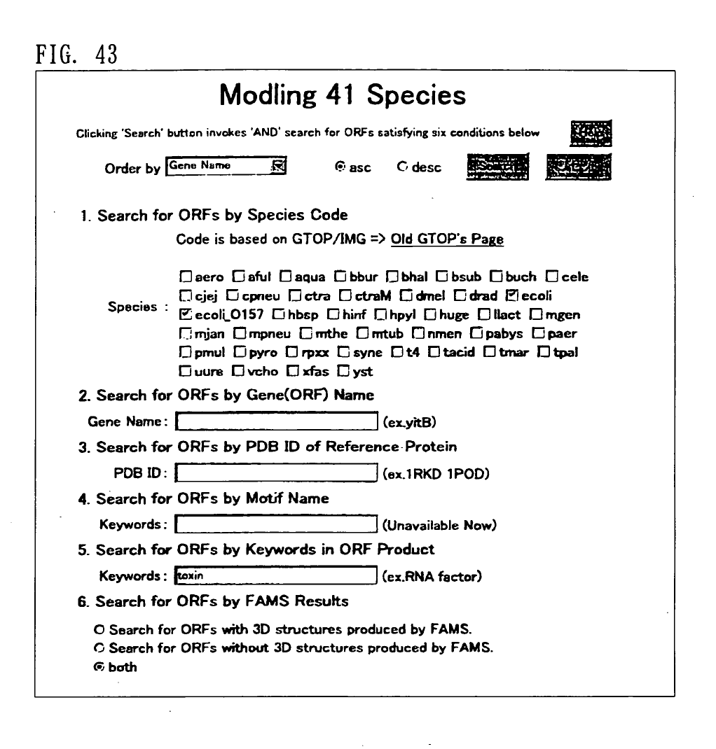

The alignment of P chain of 1BTH and I chain of 2PTC is as shown in FIG. 38. The homology was 100%.

(Alignment Result of P Chain of 1BTH and I Chain of 2PTC)

| | | | | | | |
|---|---|---|---|---|---|---|
| >1BTH_P | 58 | homology | match | mismatch | insertion | deletion |
| >2PTC_I | 58 | 100.0% | 58 | 0 | 0 | 0 |

The input file format of each alignment result for the conventional FAMS is as shown in FIG. 39.

The input file format for newFAMS this time is as shown in FIG. 40 by connecting the above-mentioned alignments with the character “U”.

As a result of the model construction only for H chain and P chain by newFAMS this time, the r.m.s.d. value of 1BTH actually registered in PDB and the 1BTH model obtained this time using newFAMS became 2.11 Å on the whole, including both H chain and P chain. Further, the r.m.s.d. value of 1BTH actually registered in PDB and the 1BTH model using the conventional FAMS was 2.18 Åon the whole. It is recognized that the r.m.s.d. value is slightly improved.

Next, it was verified whether the atoms of amino acids in a model by the conventional FAMS collide each other and the contact of amino acid residues less than 2.4 Å exists on the contact surface of H chain and P chain. As a result, the contact between atoms occurred at 7 locations between H chain and P chain molecules.

On the other hand, when it was observed whether a collision between atoms of amino acids of H chain and P chain in the model occurred by newFAMS, which is the software usable in the present invention this time, there was no collision for 2.4 Å or less.

As shown above, atoms collide each other in the conventional FAMS, however no collision between atoms could not be found in newFAMS this time. H chain of 1BTH, which is a serine protease, has a catalytic site, which is an active site, and a substance binding site in the contact region with P chain, and for the accurate description of function, the model construction of the contact region of these H chain and P chain needs to be performed precisely. In this example, an X-ray analysis was available, and being compared with it, the highly accurate construction of a temporary single chain model of plural chains was demonstrated. This indicates, again, the superiority (novelty) of newFAMS.

### EXAMPLE 5

An interface could be developed, which was designed to enable to simply and easily access the objective protein without a preliminary knowledge by conjunction search such as partial agreement of an arbitrary symbol specific to living species, a protein code name, a reference protein name, a character string of an about one-line function description for a protein, which is desired to be browsed. In FIG. 43, as an example, an interface screen for a tertiary structure model database constructed based on an alignment that can be browsed in GTOP (a home page for alignment between amino acid sequences of genome sequences publicized by National Institute of Genetics and amino acid sequences in tertiary structure database PDB) is shown. This is an interface designed to enable to simply and easily access the objective protein without a preliminary knowledge by conjunction search such as partial agreement of an arbitrary symbol specific to living species, a protein code name, a reference protein name, a character string of an about one-line function description, in the case where the tertiary structure of a protein, which is desired to be browsed, based on three-dimensional coordinates is in the above mentioned tertiary structure database of a protein of a single chain or plural chains.

In the case of this example, symbols for 41 living species, such as aero and aful, are based on the names in GTOP (as of September 2001). When there is a protein, which is desired to be browsed, according to a tertiary structure, namely a three-dimensional coordinate, a checkbox next to the symbol of the living species is checked. Further, it has a search refinement function by conjunction, such as a protein code name, a reference protein name, and an about one-line function description.

### ADVANTAGES OF THE INVENTION

According to the present invention, with respect to the prediction of a tertiary structure of a protein (including a gene encoding the protein) composed of plural chains with an unknown tertiary structure or the prediction of a tertiary structure of a conjugated protein, in which an amino acid residue of each component of the plural chains is mutated, highly reliable information of the tertiary structure of a protein including a peptide, can be obtained more efficiently and simply than by a conventional method. As a result, in the case where an interesting gene or protein is found by a genome sequence analysis, an expression profiling analysis using a DNA chip, a proteome analysis or the like, it is possible to predict a function based on the tertiary structure of the protein. Accompanied by this, it is possible to modify the peptide or the protein efficiently. In addition, by predicting the functional region of the protein, information for designing a new drug of a protein or a low molecular compound can be obtained more efficiently or effectively than by the conventional method.

### INDUSTRIAL APPLICABILITY

As mentioned above, the method of the present invention is considered extremely useful in a field in which an analysis of biological information is performed (bioinformatics) for mainly molecular design or the like of medical and agricultural chemicals. The method can extend an applicable range of a protein automatic modeling system applicable to an amino acid sequence composed of a single chain to a protein composed of plural chains, and also can diversely modify a ligand molecule, a receptor molecule, an enzyme or the like. Therefore, the usefulness of the invention is expected to further increase.

The present invention can be widely implemented in many industrial fields, in particular, fields such as drugs, foods, cosmetics, medical services, and structure analyses, therefore, it is extremely useful.

1. A method of constructing a tertiary structure of a protein composed of plural chains having given arbitrary amino acid sequences by extending an comparative modeling method of constructing a tertiary structure of a protein composed of a single chain having a given arbitrary amino acid sequence (extended modeling method),
said method comprising the steps of:
correcting an input file format of the plural chains in a computer software program so as to present a form of a temporary single chain (correction of sequence alignment), and
constructing the tertiary structure based on the modeling method while assuming that the structure has plural chains in calculation of a potential formula by the computer software program.

2. The method according to claim 1, wherein said comparative modeling method is a homology modeling method or a threading method.

3. The method according to claim 1, which is a fully automated construction method or a manual construction method.

4. The method according to claim 1, wherein said correction comprises selecting amino acid sequences of a reference protein composed of the same number of plural polypeptide chains as a target protein and adding a delimiter to the C terminal end of the amino acid sequences of the polypeptide chains, whereby enabling to handle the protein as a protein composed of a single chain (correction of sequence alignment).

5. The method according to claim 4, comprising a method of searching the reference protein from a tertiary structure database and performing sequence alignment between a target sequence and the amino acid sequence of the reference protein.

6. The method according to claim 5, wherein a software for searching the reference protein and outputting an alignment is at least one of FAMS, FASTA, PSI-BLAST, LIBRA, RPS-BLAST, IMPALA, ClustalW, HMMER and BIOCES.

7. The method according to claim 4, wherein the corrected sequence alignment is treatable to a multiple alignment, in which the amino acid sequences of the same type or a different type of reference protein is written, by using a file having a format in which an amino acid sequence of each polypeptide chain has the delimiter at the C terminal end of the amino acid sequence, and specifying a reference protein ID for each alignment section delimited by the delimiter, whereby enabling to obtain an average structure by superposing the sequences.

8. The method according to claim 1, comprising the steps of:
performing construction of Cα atomic coordinates and main-chain atomic coordinates based on the tertiary structure database and/or a database obtained by modifying the tertiary structure database so as to avoid duplication of similar structures, by determining the terminal residue number of each protein chain from the temporary single chain after the correction, disconnecting a chemical bond potential and a chemical bond angle potential at a border of the terminal residue, and adding an interatomic interaction potential at the border, and
performing minimization (optimization) of an objective function representing a temporary energy value by at least one of a simulated annealing method, a molecular dynamics calculation and a Monte Carlo method.

9. The method according to claim 1, wherein at least two chains among the plural chains constituting the target protein are polypeptide chains, and a data set of a modification whose similarity is superior or inferior is created with the use of a potential energy value as an index and based on a possible combination of 20 amino acids for each amino acid residue located at a mutual protein-protein recognition region, whereby enabling to construct the tertiary structure of the at least two polypeptide chains whose functions as respective proteins are increased or decreased.

10. The method according to claim 1, wherein, in a case where at least one chain among the plural chains constituting the target protein is an amino acid derivative or a peptide derivative (peptide ligand), and has a similar chemical structure to a corresponding ligand molecule in the reference protein, the alignment, in which the derivative of the target protein is defined as a new residue name and a one-character code and the ligand of the reference protein is defined as another new residue name and a one-character code, is created manually or automatically, and based on the possible combination of 20 amino acids and their derivatives for each residue constituting the ligand sequences, a rank is placed in the ascending order of the potential energy value, whereby enabling to construct a ligand model data set of the amino acid derivatives or the peptide derivatives, in which the plural higher-ranked sequences are stored as modifications with superior similarity to a binding region of a receptor protein.

11. The method according to claim 1, wherein at least one component of the plural chains constituting the target protein is a peptide ligand, the amino acid sequence of the ligand is fixed, and based on the possible combination of 20 amino acids for each amino acid residue located at the region recognizing the ligand, a data set of a modification with superior similarity to a binding region of the plural higher-ranked receptor proteins is created with the use of the potential energy value as an index, whereby enabling to construct a tertiary structure of various ligand receptor proteins that can bind to the ligand.

12. The method according to claim 1, wherein said plural chains are domains or modules into which a single chain peptide is divided, and are capable of being restored to a single temporary chain.

13. The method according to claim 1, wherein said target protein contains a substance, which is not included in any of a common amino acid or a peptide in which the plural common amino acids are bound, and the substance is registered in PDB or a database obtained by modifying the PDB.

14. The method according to claim 1, comprising the steps of:
searching the reference protein which is appropriate for the target sequence from the tertiary structure database and performing the sequence alignment with the amino acid sequences of the searched plural reference proteins;
selecting the amino acid sequence of the reference protein whose E-value is small for the target sequence; and
adding the delimiter to the terminal end of each amino acid sequence of the chain included in the reference protein and also adding the delimiter to the corresponding position to the target sequence (correction of sequence alignment).

15. The method according to claim 14, further comprising the steps of:
obtaining coordinates from a reference structure determined in the step of selecting the amino acid sequence of the reference protein for a Cα atom which is one of the constitutive atoms in an amino acid of the target sequence based on the alignment information and optimizing the Cα atomic coordinates;
adding a main-chain atomic coordinates from the tertiary structure database to the obtained Cα atomic coordinate and optimizing the main-chain atomic coordinates; and
adding a side-chain atomic coordinates from the tertiary structure database to the obtained main-chain atomic coordinates and optimizing the side-chain atomic coordinates.

16. The method according to claim 1, wherein said potential formula includes the following content:
in the potential formula when the total chain number=M, wherein N represents the protein chain number, k_{N} represents the serial number of the C terminal residue in the N-th protein chain, and i=1, . . . , M−1 is simplistically described as i=1, M−1,
(A) in the calculation for construction and optimization steps of the Cα atomic coordinates, a case where i=k_{N(N=1,M-1)} of a temporary chemical bond potential is not included and cases where i=k_{N(N=1,M-1)}, i=k_{N(N=1,M-1)}+1 of a temporary chemical bond angle potential are not included, and, in the case of an interatomic interaction potential, j>i+1 is added if i=k_{N}−1, and j>i is added if i=k_{N} respectively,
(B) in the calculation for construction and optimization steps of the main-chain atomic coordinates, a bond between Ci and Ni+1 is not included in a chemical bond potential when i=k_{N(N=1,M-1)}, angles Cαi-Ci-Nᵢ₊₁, Oi-Ci-Nᵢ₊₁ and Ci-Nᵢ₊₁-Cαᵢ₊₁ when i=k_{N(N=1,M-1)} wherein C and O represent a carbon atom and an oxygen atom in a carbonyl respectively, Cα represents an α carbon atom and N represents a nitrogen atom are not included in a chemical bond angle potential, further, angles Ni-Cαi-Ci-Nᵢ₊₁, Cαi-Ci-Nᵢ₊₁-Cαᵢ₊₁, and Ci-Nᵢ₊₁-Cαᵢ₊₁-Cᵢ₊₁ when i=k_{N(N=1,M-1)} are not included in a chemical bond torsional angle potential, in addition, for an interatomic interaction potential, wherein the length between atoms is represented by r, a case of rᵢⱼ ≦a specified value for rᵢⱼε{r_{Ni,Ni+1}; r_{Cαi,Ni+1}; r_{Cαi,Cαi+1}; r_{Ci,Ni+1}; r_{Ci,Cαi+1}; r_{Ci,Cβi+1}; r_{Ci,Ci+1}; r_{Oi,Ni+1}; r_{Oi,Cαi+1}} when i=k_{N(N=1,M-1)} is added.

17. A tertiary structure model of a protein, which is constructed by the method according to claim 1.

18. A database available for the extended modeling method in which data composed of the tertiary structure model of the protein constructed by the method according to claim 1, wherein a ligand model and a tertiary structure of the ligand receptor protein are fixed and combined, wherein
in said ligand model in a case where at least one chain among the plural chains constituting the target protein is an amino acid derivative or a peptide derivative (peptide ligand), and has a similar chemical structure to a corresponding ligand molecule in the reference protein, the alignment, in which the derivative of the target protein is defined as a new residue name and a one-character code and the ligand of the reference protein is defined as another new residue name and a one-character code, is created manually or automatically, and based on the possible combination of 20 amino acids and their derivatives for each residue constituting the ligand sequences, a rank is placed in the ascending order of the potential energy value, whereby enabling to construct a ligand model data set of the amino acid derivatives or the peptide derivatives, in which the plural higher-ranked sequences are stored as modifications with superior similarity to a binding region of a receptor protein, and
in said tertiary structure of the ligand receptor protein at least one component of the plural chains constituting the target protein is a peptide ligand, the amino acid sequence of the ligand is fixed, and based on the possible combination of 20 amino acids for each amino acid residue located at the region recognizing the ligand, a data set of a modification with superior similarity to a binding region of the plural higher-ranked receptor proteins is created with the use of the potential energy value as an index, whereby enabling to construct a tertiary structure of various ligand receptor proteins that can bind to the ligand.

19. A database which is constructed from data of the tertiary structure model of the protein constructed by the method according to claim 1, wherein a ligand model and a tertiary structure of the ligand receptor protein are combined so as to enable to browse or search the data by a computer, wherein
in said ligand model in a case where at least one chain among the plural chains constituting the target protein is an amino acid derivative or a peptide derivative (peptide ligand), and has a similar chemical structure to a corresponding ligand molecule in the reference protein, the alignment, in which the derivative of the target protein is defined as a new residue name and a one-character code and the ligand of the reference protein is defined as another new residue name and a one-character code, is created manually or automatically, and based on the possible combination of 20 amino acids and their derivatives for each residue constituting the ligand sequences, a rank is placed in the ascending order of the potential energy value, whereby enabling to construct a ligand model data set of the amino acid derivatives or the peptide derivatives, in which the plural higher-ranked sequences are stored as modifications with superior similarity to a binding region of a receptor protein, and
in said tertiary structure of the ligand receptor protein at least one component of the plural chains constituting the target protein is a peptide ligand, the amino acid sequence of the ligand is fixed, and based on the possible combination of 20 amino acids for each amino acid residue located at the region recognizing the ligand, a data set of a modification with superior similarity to a binding region of the plural higher-ranked receptor proteins is created with the use of the potential energy value as an index, whereby enabling to construct a tertiary structure of various ligand receptor proteins that can bind to the ligand.

20. A database structure characterized in that the following content can be browsed or searched by a computer:
a gene identification code or a protein identification code of a target protein composed of plural chains, an about one-line function description, a target amino acid sequence and coordinates of three-dimensional structure of the target protein;
a gene identification code or a protein identification code of a reference protein, an about one-line function description, a reference amino acid sequence and coordinates of three-dimensional structure of the reference protein; and
an alignment result between the target sequence and the reference sequence, a homology value and an E-value.

21. A computer software program being capable of browsing or searching the content or using the structure of the database according to claim 18, or a computer installed therewith.

22. An interface being designed to enable to access a target protein by conjunction search such as partial agreement of an arbitrary symbol specific to living species, a protein code name, a reference protein name, a character string of an about one-line function description for a protein, which is desired to be browsed, among the tertiary structure database constructed by the method according to claim 1.

23. The method according to claim 10, wherein said amino acid derivative is a non-natural amino acid such as βAsp or γGlu or a derivative thereof.

24. A program (newFAMS) comprising the method according to claim 1 or a computer installed therewith.

25. The method according to claim 1, wherein said target protein composed of plural chains includes one or more polypeptide chains.

26. The method according to claim 25, wherein at least one chain among the plural chains is selected from the group consisting of natural or non-natural amino acids and amino acid derivatives such as their derivatives, peptide derivatives, pharmaceutical substances, nucleic acids, saccharides, organic metal compounds, metal oxides and their ions, metals and their ions.

27. The method according to claim 1, comprising the steps of, for the target protein and the selected reference protein, assuming each amino acid sequence of the plural chains included in the target protein and the selected reference protein respectively as a single chain in a state where the N terminal ends and the C terminal ends are bound sequentially, performing sequence alignment between the reference sequence of the thus obtained temporary single chain and the target sequence of the thus obtained temporary single chain to confirm their correspondence relationship, locating a Cα atom, which is one of the constitutive atoms in the amino acid residue in the target sequence, binding the Cα atoms by an amide bond, further adding a side-chain to construct coordinates for other constitutive atoms, performing optimization, and constructing the tertiary structure of the target protein by the modeling method.

28. The method according to claim 1, wherein the construction of the tertiary structure of the target protein is performed by obtaining the coordinates from the tertiary structure of the reference protein selected for the Cα atom in a main-chain amino acid in the target protein based on the obtained alignment information, optimizing the Cα atomic coordinates so as to minimize an objective function, adding coordinates of other atoms (including a Cβ atomic coordinates) of the main-chain to the optimized Cα atomic coordinates, optimizing the main-chain atomic coordinates so as to minimize the objective function, adding coordinates of other atoms of the side-chain to the optimized main-chain atomic coordinates, and optimizing the side-chain atomic coordinates so as to minimize the objective function.

29. The method according to claim 27, wherein the construction of the tertiary structure of the target protein is performed by obtaining the coordinates from the tertiary structure of the reference protein selected for the Cα atom in a main-chain amino acid in the target protein based on the obtained alignment information, optimizing the Cα atomic coordinates so as to minimize an objective function, adding coordinates of other atoms (including a Cβ atomic coordinates) of the main-chain to the optimized Cα atomic coordinates, optimizing the main-chain atomic coordinates so as to minimize the objective function, adding coordinates of other atoms of the side-chain to the optimized main-chain atomic coordinates, and optimizing the side-chain atomic coordinates so as to minimize the objective function.

30. A computer software program being capable of browsing or searching the content or using the structure of the database to claim 19, or a computer installed therewith.

31. A computer software program being capable of browsing or searching the content or using the structure of the database according to claim 20, or a computer installed therewith.


SEQUENCE LISTING (13 sequences)

SEQ ID NO: 1 - protein, 1199 aa, Rattus norvegicus
MVRLLLIFFPMIFLEMSILPRMPDRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGLNRCLPDGQTLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKKVCTGNESLEENYVQDSKMGFVINAIYAMAHGLQNMHHALCPGHVGLCDAMKPIDGRKLLDFLIKSSFVGVSGEEVWFDEKGDAPGRYDIMNLQYTEANRYDYVHVGTWHEGVLNIDDYKIQMNKSGMVRSVCSEPCLKGQIKVIRKGEVSCCWICTACKENEFVQDEFTCRACDLGWWPNAELTGCEPIPVRYLEWSDIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPVGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKPGAGNANSNGKSVSWSEPGGRQAPKGQHVWQRLSVHVKTNETACNQTAVIKPLTKSYQGSGKSLTFSDASTKTLYNVEEEDNTPSAHFSPPSSPSMVVHRRGPPVATTPPLPPHLTAEETPLFLADSVIPKGLPPPLPQQQPQQPPPQQPPQQPKSLMDQLQGVVTNFGSGIPDFHAVLAGPGTPGNSLRSLYPPPPPPQHLQMLPLHLSTFQEESISPPGEDIDDDSERFKLLQEFVYEREGNTEEDELEEEEDLPTASKLTPEDSPALTPPSPFRDSVASGSSVPSSPVSESVLCTPPNVTYASVILRDYKQSSSTL

SEQ ID NO: 2 - protein, 1203 aa, Rattus norvegicus
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLRSHLPKARVVACFCEGMTVRGLLMAMRRLGLAGEFLLLGSDGWADRYDVTDGYQREAVGGITIKLQSPDVKWFDDYYLKLRPETNLRNPWFQEFWQHRFQCRLEGFAQENSKYNKTCNSSLTLRTHHVQDSKMGFVINAIYSMAYGLHNMQMSLCPGYAGLCDAMKPIDGRKLLDSLMKTNFTGVSGDMILFDENGDSPGRYEIMNFKEMGKDYFDYINVGSWDNGELKMDDDEVWSKKNNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVIFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSTRGQHLWQRLSVHINKKENPNQTAVIKPFPKSTENRGPGAAAGGGSGPGVAGAGNAGCTATGGPEPPDAGPKALYDVAEAEESFPAAARPRSPSPISTLSHLAGSAGRTDDDAPSLHSETAARSSSSQGSLMEQISSVVTRFTANISELNSMMLSTAATPGPPGTPICSSYLIPKEIQLPTTMTTFAEIQPLPAIEVTGGAQGATGVSPAQETPTGAESAPGKPDLEELVALTPPSPFRDSVDSGSTTPNSPVSESALCIPSSPKYDTLIIRDYTQSSSSL

SEQ ID NO: 3 - protein, 474 aa, Rattus norvegicus
RRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLRSHLPKARVVACFCEGMTVRGLLMAMRRLGLAGEFLLLGSDGWADRYDVTDGYQREAVGGITIKLQSPDVKWFDDYYLKLRPETNLRNPWFQEFWQHRFQCRLEGFAQENSKYNKTCNSSLTLRTHHVQDSKMGFVINAIYSMAYGLHNMQMSLCPGYAGLCDAMKPIDGRKLLDSLMKTNFTGVSGDMILFDENGDSPGRYEIMNFKEMGKDYFDYINVGSWDNGELKMDDDEV

SEQ ID NO: 4 - protein, 456 aa, Rattus norvegicus
QRSVARMDGDVIIGALFSVHHQPPAEKVPERKCGEIREQYGIQRVEAMFHTLDKINADPVLLPNITLGSEIRDSCWHSSVALEQSIEFIRDSLISIRKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKKVCTGNESLEENYVQDSKMGFVINAIYAMAHGLQNMHHALCPGHVGLCDAMKPIDGRKLLDFLIKSSFVGVSGEEVWFDEKGDAPGRYDIMNLQYTEANRYDYVHVGTWHEGVLNIDDYKI

SEQ ID NO: 5 - protein, 555 aa, Escherichia coli
WQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHND

SEQ ID NO: 6 - protein, 573 aa, Gallus gallus
LFRYSSCTDKLLMIFGSLLAIAHGTSLPIAMIIFGDMTDSFVTSGMTNITGNSSGLNSSADVFNKLEEEMTRYAYYYSAIAAAVLVAAYIQTSFWTLAAGRQVKKIREKFFHAIMRQEIGWFDVNDAGELNTRLIDDVSKINEGIGDKIGFLIQSETTFLTGFIVGFIRGWKLTLVILAVSPVLGLSAALWAKILTAFTDKEQAAYAKAGAVAEEVLSAVRTVIAFGGQEKEIKRYHKNLEDAKRIGIRKAITSNISMGAAFLLIYASYALAFWYGTTLILANEYSIGNVLTVFFSVLIGAFSIGQTAPSIEAFANARGAAYAIFNIIDNEPEIDSYSDAGHKPDHIKGNLEFQNVFFNYPSRPDVEILKGLNLKVNCGQTVALVGGSGCGKSTTVQLIQRFYDPKEGTITIDGQDLKSLNVRYLREIIGVVNQEPVLFATTIAENIRYGREDVTMEEIERATKEANAYDFIMKLPKKFETVVGERGAQMSGGQKQRIAIARALVHNPKILLLDEATSALDTESESVVQAALDKAREGRTTVVVAHRLSTVRNADLIAVFESGVITEQGNHSQ

SEQ ID NO: 7 - protein, 549 aa, Escherichia coli
LWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHND

SEQ ID NO: 8 - protein, 748 aa, Bacillus anthracis
NKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 9 - protein, 16 aa, Bacillus anthracis
MLARRKPVLPALTINP

SEQ ID NO: 10 - protein, 16 aa, Bacillus anthracis
MFFRRKPVLPALTINP

SEQ ID NO: 11 - protein, 254 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTTNVGKGQPSVLQVVNLPIVERPVCKDSTRIRITDNMFCAGYKPDEGKRGDACQGDSGGPFVMKSPFNNRWYQMGIVSWGEGCDRDGKYGFYTHVFRLKKWIQKVI

SEQ ID NO: 12 - protein, 58 aa, Bos taurus [sequence omitted]

SEQ ID NO: 13 - protein, 223 aa, Bos taurus
IVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN